(19) United States
(12) Patent Application Publication
(10) Pub. No.: US 2025/0160912 A1
(43) Pub. Date: May 22, 2025

(54) System and Method for Bone Fracture Repair

(71) Applicant: Dunamis Medical Technologies, LLC, Greenville, AL (US)

(72) Inventors: Prithviraj Chavan, Greenville, AL (US); Forrest Samuel, Carlsbad, CA (US)

(21) Appl. No.: 18/950,070

(22) Filed: Nov. 16, 2024

Related U.S. Application Data
(60) Provisional application No. 63/599,795, filed on Nov. 16, 2023.
(60) Provisional application No. 63/601,128, filed on Nov. 20, 2023.

(51) Int. Cl. A61B 17/82; A61B 17/84; A61B 17/86

(52) U.S. Cl. CPC A61B 17/82; A61B 17/844; A61B 17/864

(57) ABSTRACT

A bone fracture repair system using a tension/compression lock configuration is described herein. In some embodiments, the bone fracture repair system includes one or more bone anchors, one or more locking elements configured to nest within the one or more bone anchors, and one or more flexible tension members configured to interact with the bone anchors and locking elements to securely hold fractured bone fragments in place for healing, while also creating a tension band construct that prevents displacement of the bone fragments by forces that tend to pull the bone fragments apart.

### CROSS-REFERENCES TO RELATED APPLICATIONS

The present application is a nonprovisional application claiming the benefit of priority under 35 U.S.C. § 119(e) from U.S. Provisional Application No. 63/599,795, filed on Nov. 16, 2023, and U.S. Provisional Application No. 63/601,128, filed on Nov. 20, 2023, the complete disclosures of which is hereby incorporated by reference into this disclosure as if set forth fully herein.

### FIELD

The present disclosure relates generally to soft tissue-to-bone and bone-to-bone repair reconstruction, and more particularly to a system and method for repairing fractured bone using a plurality of bone anchors and one or more tension members secure the repair construct.

### BACKGROUND

Current tension band repairs require either the use of stainless-steel wire or suture tape configurations. Metal wires have a high revision rate as seen with patella fractures. Its use results in a high number of revisions due to implant related symptoms. A recent technique of using a tape as a substitute to stainless steel wires requires surgeons to tie a knot. Several challenges with this technique include knot failure and the need for maintaining the repair under tension when the knot is tied.

Patella fractures typically account for 1% of skeletal fractures and would account for around 350,000-400,000 repairs performed every year. They have a high revision rate related to hardware problems. Fractures that imperfectly unite can also cause arthritis and require resurfacing arthroplasty. The novel technology disclosed herein will optimize the repair by allowing for precise tensioning, avoiding usage of metal hardware to perform a tension band repair, and minimizing loosening which can cause distraction of the fracture site.

### SUMMARY

The present disclosure introduces a novel technique when performing this repair (i.e. tension band construct) which allows for the repair to be performed in a knotless manner. In addition to bone fracture repair, this technique may be used for soft tissue to bone and bone to bone repairs. Although shown and described herein in relation to a patella fracture, it should be understood that the bone fracture repair system disclosed herein may be used to repair soft tissue and/or fractured bones in any suitable anatomical location in the body, including but not limited to patella, ankle, medial malleolus, olecranon, and the like.

Once installed, the bone fracture repair system of the present disclosure works to reduce the fracture and maintain compression of the fractured bone to promote healing. By way of example, reduction is maintained by: a) tension created in the system, b) the locking interface, and c) a counter force which is created by the looped tension member, which based on its interaction with the locking element counteracts forces that try to separate the fracture by increasing tension in the system. These features will create resistance to any forces that try to create distraction and separation of fragments that have been repaired. Using this novel technique, the surgeons will be able to create tension in the system and perform a tension band repair of fractured bone. Once assembled, the tension members are able to slide in one direction allowing the surgeon to take away any slack (and/or increase the tension) in the system and once maximum tension is achieved the system will lock and perform repair in a knotless manner.

By way of example only, the various embodiments disclosed herein demonstrate the repair performed in a crisscross manner, but it is possible to perform the repair with a single screw and locking element configuration while trapping the tension member at the locking interface.

In some embodiments, the bone fracture repair system described herein includes at least one bone anchor, at least one locking element, and at least one flexible tension member configured to interact with the bone anchor and locking element to securely hold fractured bone fragments in place for healing, while also creating a tension band construct that prevents displacement of the bone fragments by forces that tend to pull the bone fragments apart.

In some embodiments, the bone anchor has a head at a proximal end, an elongated shank extending distally from the head, and an interior lumen or cannulation extending the length of the shank and having a proximal opening within the head and a distal opening at the distal end of the shank. In some embodiments, the head further includes a proximal recess including a drive feature configured to engage with a driver tool. In some embodiments, the head may further include one or more flutes formed in the outer surface to ease insertion of the head into bone. In some embodiments, the shank comprises a cylindrical member having a smooth outer surface portion and a threaded outer surface portion. In some embodiments, the threaded outer surface portion is located at or near the distal end of the shank, and the smooth outer surface portion is located near the head. In some embodiments, the bone anchor may have a fully threaded shank. By way of example only, the threaded outer surface portion is configured for insertion into a bone fragment (e.g., the distal bone fragment of the fracture) and gain purchase within the distal bone fragment to hold the distal and proximal bone fragments together. In some embodiments, the head further includes a circumferential mating surface which may be a tapered or concave surface positioned within the proximal recess and surrounding the proximal opening of the shank cannulation. In some embodiments, the circumferential mating surface operates as a first locking surface that cooperates with the distal engagement surface and/or a concave cutout positioned within the distal engagement surface of the locking element (e.g. second locking surfaces) to form one or more “pinch points” that capture the tension member therebetween thereby locking the bone fracture repair system under tension. By way of example, the bone anchor may be provided in any suitable length. In some embodiments, the bone anchor may have a shank without a head, and the “pinch point” may be formed at a proximal end of the headless shank for example at a proximal opening of the cannulation or at a location within the cannulation.

In some embodiments, the locking element has a proximal head and a distal extension extending longitudinally from the proximal head. In some embodiments, the locking element is configured such that the distal extension is sized and configured to extend into the cannulation of the bone anchor while the head is received within the proximal recess.

In some embodiments, the proximal head includes an interior cavity and a plurality of apertures formed in the head that function as ingress and egress ports for the tension member to pass through during assembly and tensioning of the bone fracture repair system. For example, in some embodiments the head may have a proximal aperture and a pair of transverse apertures positioned on opposite sides of the head. In some embodiments, the proximal head has a bottom or distal portion including a distal engagement surface that may be convex or tapered and may or may not include a concave cutout positioned within the distal engagement surface. In some embodiments, the distal engagement surface and/or concave cutout operate as a second locking surface that cooperates with the circumferential mating surface of the bone anchor (e.g., the first locking surface) to form one or more “pinch points” that capture the tension member therebetween thereby locking the bone fracture repair system under tension. By way of example, the concave cutout provides a space for the tension member to pass between the bone anchor and plug insert while still being captured and locked within the concave cutout upon final tightening of the system. In some embodiments, the proximal head may include one or more smooth concave lateral recesses positioned near the transverse apertures and that are configured to provide clearance for the tension members to pass during use. In some embodiments, the proximal head may include one or more smooth concave proximal surfaces positioned near the proximal aperture and are configured to provide a smooth contact surface for the tension members as the tension members pass through the head during use.

In some embodiments, the distal extension comprises an elongated body having an oblong shape and including a pair of opposing long sides and a pair of opposing short sides. In some embodiments, the short sides are curved and configured to contact or engage with the inner wall of the cannulation of the bone anchor to prevent a toggle motion of the plug insert while engaged with the bone anchor. In some embodiments, the long sides are flat or planar and are included to provide space within the cannulation for the tension members to travel through. In some embodiments, the long sides have a convex curvature. In some embodiments, the long sides have a concave curvature.

In some embodiments, the distal extension may include an open docking slot that is open to one of the curved sides. In some embodiments, the open docking slot may include an angled proximal surface angling proximally to one of the curved sides (to create the open slot), a hooked end, and smooth rounded surfaces configured to reduce the effect of friction on the tension members as the tension members are moved within the open docking slot during assembly and/or tightening.

As used throughout this disclosure, the tension member is defined has having a looped portion (or “looped end”) and a pair of free ends. By way of example, the looped portion is defined as the portion of the tension member that ultimately engages with (and “loops around”) the docking slot of the locking element. The free ends are the actual ends of the tension member.

By way of example, the bone fracture repair system of the present disclosure may be assembled and used in several configurations to achieve the same goal of creating a tension band repair construct that knotlessly locks the repair under tension.

In some embodiments, a one example of assembly and use of the bone fracture repair system includes a pair of bone anchors, a single locking element having an open docking slot, and a single tension member. As a first step, the user (e.g., surgeon) positions the bone anchors through a fractured bone (e.g., patella). At this point, the locking element is not coupled to either of the bone anchors. In some embodiments, the locking element and tension member are coupled by passing the free ends of the tension member through the proximal and transverse apertures of the locking element in a distal direction such that the free ends are positioned distally of the locking element and a looped portion is formed proximal of the head.

Once the locking element and tension member are coupled, the locking element is positioned in the vicinity of the proximal head of the first bone anchor. At this point, the free ends may be shuttled from the proximal end of the bone anchor to the distal end of the bone anchor. The looped portion is then passed on the anterior surface of the bone from the proximal head of the locking element to the distal end of the second bone anchor, then shuttled proximally through the second bone anchor, passed over the anterior surface of the bone (e.g., in a crisscross or “X” pattern) to the distal end of the first bone anchor, and shuttled proximally through the first bone anchor to the locking element which is located proximal of the bone anchor head. The looped portion is then coupled with the locking element by passing the looped portion into the open docking slot. Once this coupling has been established, the user can begin to tighten the system by pulling on the free ends of the tension member and/or the portion of tension member exiting from the distal end and spanning across the bone to the proximal end of the second bone anchor. This will cause the locking element to translate in a distal direction to pull the locking element into the cannulation of the first bone anchor. In some embodiments, this can also be achieved by manually pushing the locking element distally. Once the locking element is seated in the bone anchor, the tension member may be tensioned by pulling the free ends in a distal direction one at a time or simultaneously. This will remove the slack out of the system, drawing the head of the locking element further into the proximal recess of the head of the bone anchor, creating the “pinch points” described above between the distal engagement surface and/or concave recess of the locking element and the circumferential mating surface of the bone anchor with the tension member captured between. This will create a tension band construct which can prevent against displacement of the fracture created by forces that tend to pull the fracture fragments apart.

In some embodiments, another example of assembly and use of the bone fracture repair system includes a pair of bone anchors, two locking elements having open docking slots, and two tension members. As a first step, the user (e.g., surgeon) positions the bone anchors through a fractured bone (e.g., patella). At this point, the locking elements are not coupled to either of the bone anchors. In some embodiments, the locking elements and tension members are coupled by passing the free ends of the tension members through the proximal and transverse apertures of the respective locking elements in a distal direction, such that the free ends are positioned distally of the locking elements and looped portions is formed proximal of the heads.

Once the locking element and tension member are coupled, the locking elements are positioned in the vicinity of the proximal heads of the first and second bone anchors. At this point, the free ends may be shuttled from the proximal end of the bone anchors to the distal ends of the respective bone anchors. The looped portions are then passed on the anterior surface of the bone from the proximal heads of the respective locking elements to the distal ends of the opposite bone anchors, then shuttled proximally through the bone anchors and coupled with the locking elements by passing the looped portions into the respective open docking slots. Once these couplings have been established, the user can begin to tighten the system by pulling on the free ends of the tension members and/or the portions of tension members exiting from the distal ends and spanning across the bone to the proximal ends of the opposite bone anchors. This will cause the locking elements to translate in a distal direction to pull the locking elements into the bone anchors. In some embodiments, this can also be achieved by manually pushing the locking elements distally. Once the locking elements are seated in the respective bone anchors, the tension members may be tensioned by pulling the free ends in a distal direction one at a time or simultaneously. This will remove the slack out of the system, drawing the heads of the locking elements further into the proximal recesses of the heads of the bone anchors, creating the “pinch points” described above between the distal engagement surfaces and/or concave recesses of the locking elements and the circumferential mating surfaces of the bone anchor with the tension members captured between. This will create a tension band construct which can prevent against displacement of the fracture created by forces that tend to pull the fracture fragments apart.

In some embodiments, another example of assembly and use of the bone fracture repair system includes a single bone anchor, a single locking element having an open docking slot, and a single tension member. As a first step, the user (e.g., surgeon) positions the bone anchor through a fractured bone (e.g., patella), and forms a bone tunnel spanning the fracture near the bone anchor. At this point, the locking element is not coupled to the bone anchor. In some embodiments, the locking element and tension member are coupled by passing the free ends of the tension member through the proximal and transverse apertures of the locking element in a distal direction such that the free ends are positioned distally of the locking element and a looped portion is formed proximal of the head.

Once the locking element and tension member are coupled, the locking element is positioned in the vicinity of the proximal head of the bone anchor. At this point, the free ends may be shuttled from the proximal end of the bone anchor to the distal end of the bone anchor. The looped portion is then passed on the anterior surface of the bone from the proximal head of the locking element to the distal opening of the bone tunnel, then shuttled proximally through the bone tunnel, passed over the anterior surface of the bone (e.g., in a crisscross or “X” pattern) to the distal end of the bone anchor, and shuttled proximally through the bone anchor to the locking element which is located proximal of the bone anchor head. The looped portion is then coupled with the locking element by passing the looped portion into the open docking slot. Once this coupling has been established, the user can begin to tighten the system by pulling on the free ends of the tension member and/or the portion of tension member exiting from the distal end and spanning across the bone to the proximal opening of the bone tunnel. This will cause the locking element to translate in a distal direction to pull the locking element into the cannulation of the first bone anchor. In some embodiments, this can also be achieved by manually pushing the locking element distally. Once the locking element is seated in the bone anchor, the tension member may be tensioned by pulling the free ends in a distal direction one at a time or simultaneously. This will remove the slack out of the system, drawing the head of the locking element further into the proximal recess of the head of the bone anchor, creating the “pinch points” described above between the distal engagement surface and/or concave recess of the locking element and the circumferential mating surface of the bone anchor with the tension member captured between. This will create a tension band construct which can prevent against displacement of the fracture created by forces that tend to pull the fracture fragments apart.

In some embodiments, another example of assembly and use of the bone fracture repair system includes a pair of bone anchors, a single locking element having a closed docking slot, and a single tension member. In some embodiments, the same overall assembly may be achieved by chaperoning the free ends of the tension member through the bone anchors (as an alternative to chaperoning the looped end as described above). As a first step, the user (e.g., surgeon) is positions a pair of bone anchors through a fractured bone. At this point, the locking element is not coupled to either of the bone anchors. In some embodiments, the locking element and tension member are coupled by first passing the tension member through the closed docking slot and allowing the tension member to settle such that the free ends are positioned distally of the locking element and a looped portion is formed within the closed docking slot.

Once the locking element and tension member are coupled (or provided as pre-coupled), the locking element is positioned in the vicinity of the proximal head of the first bone anchor. At this point, the free ends may be shuttled from the proximal end of the first bone anchor to the distal end of the first bone anchor. The free ends are then passed on the anterior surface of the bone from the distal end of the first bone anchor to the proximal end of the second bone anchor, then shuttled distally through the second bone anchor. Once the free ends exit the distal opening of the second bone anchor, they are then passed on the anterior surface of the bone (e.g., in a crisscross or “X” pattern) to the locking element. Assembly continues by passing the free ends of the tension member through the proximal aperture and the transverse apertures of the locking element and through first bone anchor such that the free ends are positioned distal of the first bone anchor.

Once this assembly has been established, the user can begin to tighten the system by pulling on the free ends of the tension member. This will cause the locking element to translate in a distal direction into the first bone anchor. This can also be achieved by manually pushing the locking element distally. Once the locking element is seated in the bone anchor, the tension member may be tensioned by pulling the free ends and/or the loop component exiting from the distal end of the first bone anchor in a distal direction one at a time (e.g., sequentially) or simultaneously. This will remove the slack out of the system, drawing the head of the locking element further into the proximal recess of the head of the bone anchor, creating the “pinch points” described above between the distal engagement surface and/or concave cutout of the locking element and the circumferential mating surface of the bone anchor with the tension member captured between. This will create a tension band construct which can prevent against displacement of the fracture created by forces that tend to pull the fracture fragments apart.

In some embodiments, another example of assembly and use of the bone fracture repair system includes a pair of bone anchors, a pair of locking elements having a closed docking slot, and a pair of tension members. As a first step, the user (e.g., surgeon) is positions a pair of bone anchors through a fractured bone. At this point, the locking elements are not coupled to either of the bone anchors. In some embodiments, the locking elements and tension members are coupled by first passing the tension members through the closed docking slot of the respective locking elements and allowing the tension members to settle such that the free ends are positioned distally of the locking element and a looped portion is formed within the closed docking slot.

Once the locking elements and tension members are coupled, the locking elements are positioned in the vicinity of the proximal heads of the bone anchors. At this point, the free ends may be shuttled distally through the bone anchors. The free ends are then passed on the anterior surface of the bone from the distal ends of their respective bone anchors to the proximal ends of the opposite locking elements, then shuttled through the locking element heads and respective bone anchors such that the free ends extend distally from the respective bone anchors.

Once this assembly has been established, the user can begin to tighten the system by pulling on the free ends of the tension members. This will cause the locking elements to translate in a distal direction into their respective bone anchors. This can also be achieved by manually pushing the locking elements distally. Once the locking elements are seated in the bone anchors, the tension members may be tensioned by pulling the free ends and/or the loop components exiting from the distal end of the first bone anchor in a distal direction one at a time or simultaneously. This will remove the slack out of the system, drawing the heads of the locking element further into the proximal recesses of the heads of the bone anchors, creating the “pinch points” described above between the distal engagement surfaces and/or concave cutouts of the locking elements and the circumferential mating surfaces of the bone anchors with the tension members captured between. This will create a tension band construct which can prevent against displacement of the fracture created by forces that tend to pull the fracture fragments apart.

In some embodiments, another example of assembly and use of the bone fracture repair system includes a single of bone anchor, a single locking element having a closed docking slot, and a single tension member. As a first step, the user (e.g., surgeon) is positions a bone anchor through a fractured bone and forms a bone tunnel extending across the fracture in the bone near the bone anchor. At this point, the locking element is not coupled to the bone anchor. In some embodiments, the locking element and tension member are coupled by first passing the tension member through the closed docking slot and allowing the tension member to settle such that the free ends are positioned distally of the locking element and a looped portion is formed within the closed docking slot.

Once the locking element and tension member are coupled (or provided as pre-coupled), the locking element is positioned in the vicinity of the proximal head of the bone anchor. At this point, the free ends may be shuttled from the proximal end of the bone anchor to the distal end of the bone anchor. The free ends are then passed on the anterior surface of the bone from the distal end of the bone anchor to the proximal opening of the bone tunnel, then shuttled distally through the bone tunnel. Once the free ends exit the distal opening of the bone tunnel, they are then passed over the anterior surface of the bone (e.g., in a crisscross or “X” pattern) to the locking element. Assembly continues by passing the free ends of the tension member through the proximal aperture and the transverse apertures of the locking element and through bone anchor such that the free ends are positioned distal of the bone anchor.

Once this assembly has been established, the user can begin to tighten the system by pulling on the free ends of the tension member and/or the loop component exiting from the distal end of the bone anchor. This will cause the locking element to translate in a distal direction into the bone anchor. This can also be achieved by manually pushing the locking element distally. Once the locking element is seated in the bone anchor, the tension member may be tensioned by pulling the free ends in a distal direction one at a time (e.g., sequentially) or simultaneously. This will remove the slack out of the system, drawing the head of the locking element further into the proximal recess of the head of the bone anchor, creating the “pinch points” described above between the distal engagement surface and/or concave cutout of the locking element and the circumferential mating surface of the bone anchor with the tension member captured between. This will create a tension band construct which can prevent against displacement of the fracture created by forces that tend to pull the fracture fragments apart.

In some embodiments, several examples of assembly and use of the bone fracture repair system are nearly identical to the closed docking slot examples presented above, except that the locking of the tension members occurs between a tapered outer surface of the locking element and tapered inner surface of cannulated bone anchors. By way of example, the locking under tension occurs by a wedging interaction instead of a pinch point interaction.

In any of the described embodiments, locking is maintained by the tension created in the construct as well as the looped portion of the tension member which is docked into the open docking slot that continues to assist with preventing any separation of the fracture by providing a counter force in the opposite direction. If the user desires to revise the tension or if revision surgery proves necessary, the locking element can be loosened by passing a counterforce suture (for example) through the transverse apertures and pulling the locking element in a proximal direction. In some embodiments, the counterforce suture i may be preloaded into the locking element. In some embodiments, the counterforce suture may be guided through the transverse apertures before final tensioning of the system.

As additional description to the embodiments described below, the present disclosure describes the following embodiments.

Embodiment 1 is a bone fracture repair system, comprising: a flexible tension member having first and second free ends and a flexible body extending between the first and second free ends, the flexible tension member oriented in an assembly configuration having a looped end comprising a U-shaped portion of the flexible body and an open end comprising the first and second free ends of the flexible tension member; first and second bone anchors configured for implantation into bone, each of the first and second bone anchors having an anchor head at a proximal end, an elongated shank extending between the anchor head and a distal end, and a cannulation extending longitudinally through the elongated shaft and having a proximal opening at the proximal end and a distal opening at the distal end, the anchor head comprising a proximal recess including a circumferential mating surface positioned within the proximal recess; a locking element having a proximal head and an elongated extension extending distally from the proximal head, the proximal head including a proximal opening, a pair of transverse openings, and a distal engagement surface, the elongated extension including a docking slot formed therein and configured to capture the looped end of the flexible tension member; a first assembly configuration in which the locking element is associated with the first bone anchor, the looped end of the flexible tension member is positioned proximal of the locking element, the open end of the flexible tension member is positioned distal of the distal end of the first bone anchor, and the flexible body extends through the proximal opening of the locking element, the transverse openings of the locking element, and the cannulation of the first bone anchor; and a second assembly configuration in which the elongated extension of the locking element extends into the cannulation of the first bone anchor, the looped end of the flexible tension member is captured by the docking slot of the locking element, the open end of the flexible tension member is positioned distal of the distal end of the first bone anchor, and the flexible body extends through the cannulation of the first bone anchor, the cannulation of the second bone anchor, and the proximal and transverse openings of the locking element before passing between the circumferential mating surface of the first bone anchor and the distal engagement surface of the locking element; wherein the open end of the flexible tension member is configured to be pulled taut in the distal direction, thereby creating tension in the flexible tension member which causes the locking element to translate distally relative to the first bone anchor and transition the bone fracture repair system from an unlocked state that permits movement of the flexible tension member to a locked state that prohibits movement of the flexible tension member, where the locked state is maintained via compression and friction applied to the flexible tension member between the mating surface of the first bone anchor and the engagement surface of the locking element.

Embodiment 2 is the bone fracture repair system of embodiment 1, wherein the proximal head of the locking element is configured to nest within the anchor head.

Embodiment 3 is the bone fracture repair system of embodiments 1 or 2, wherein the distal engagement surface of the locking element includes at least one concave recess.

Embodiment 4 is the bone fracture repair system of any of embodiments 1 through 3, wherein the second assembly configuration further comprises the flexible body extending distally from the looped end through the cannulation of the first bone anchor, then diagonally to the proximal end of the second bone anchor, then distally through the cannulation of the second bone anchor, then diagonally to the proximal head of the locking element, and then distally through the proximal opening of the locking element, the transverse openings of the locking element, and the cannulation of the first bone anchor.

Embodiment 5 is the bone fracture repair system of any of embodiments 1 through 4, wherein the shanks of the first and second bone anchors have outer surfaces comprising a threaded portion and a non-threaded portion.

Embodiment 6 is the bone fracture repair system of any of embodiments 1 through 5, wherein the locking element further includes at least one smooth curved surface bordering the docking slot.

Embodiment 7 is the bone fracture repair system of any of embodiments 1 through 6, wherein docking slot is an open docking slot.

Embodiment 8 is the bone fracture repair system of any of embodiments 1 through 7, wherein the docking slot has a hook configuration

Embodiment 9 is the bone fracture repair system of any of embodiments 1 through 8, wherein the docking slot extends to the distal end of the elongated extension.

Embodiment 10 is the bone fracture repair system of any of embodiments 1 through 9, wherein the elongated extension is sized and configured to snugly nest within the cannulation of the first bone anchor to prevent pivoting movement of the locking element when the elongated extension is positioned within the cannulation of the first bone anchor.

Embodiment 11 is a bone fracture repair system, comprising: a flexible tension member having first and second free ends and a flexible body extending between the first and second free ends, the flexible tension member oriented in an assembly configuration having a looped end comprising a U-shaped portion of the flexible body and an open end comprising the first and second free ends of the flexible tension member; a bone anchor configured for implantation into bone, the bone anchor having an anchor head at a proximal end, an elongated shank extending between the anchor head and a distal end, and a cannulation extending longitudinally through the elongated shaft and having a proximal opening at the proximal end and a distal opening at the distal end, the anchor head comprising a proximal recess including a circumferential mating surface positioned within the proximal recess; a locking element having a proximal head and an elongated extension extending distally from the proximal head, the proximal head including a proximal opening, a pair of transverse openings, and a distal engagement surface, the elongated extension including a docking slot formed therein and configured to capture the looped end of the flexible tension member; a first assembly configuration in which the locking element is associated with the bone anchor, the looped end of the flexible tension member is positioned proximal of the locking element, the open end of the flexible tension member is positioned distal of the distal end of the bone anchor, and the flexible body extends through the proximal opening of the locking element, the transverse openings of the locking element, and the cannulation of the bone anchor; and a second assembly configuration in which the elongated extension of the locking element extends into the cannulation of the bone anchor, the looped end of the flexible tension member is captured by the docking slot of the locking element, the open end of the flexible tension member is positioned distal of the distal end of the bone anchor, and the flexible body extends through the cannulation of the bone anchor, a bone tunnel formed in the fractured bone under repair, and the proximal and transverse openings of the locking element before passing between the circumferential mating surface of the bone anchor and the distal engagement surface of the locking element; wherein the open end of the flexible tension member is configured to be pulled taut in the distal direction, thereby creating tension in the flexible tension member which causes the locking element to translate distally relative to the bone anchor and transition the bone fracture repair system from an unlocked state that permits movement of the flexible tension member to a locked state that prohibits movement of the flexible tension member, where the locked state is maintained via compression and friction applied to the flexible tension member between the mating surface of the bone anchor and the engagement surface of the locking element.

Embodiment 12 is the bone fracture repair system of embodiment 11, wherein the proximal head of the locking element is configured to nest within the anchor head.

Embodiment 13 is the bone fracture repair system of embodiments 11 or 12, wherein the distal engagement surface of the locking element includes at least one concave recess.

Embodiment 14 is the bone fracture repair system of any of embodiments 11 though 13, wherein the second assembly configuration further comprises the flexible body extending distally from the looped end through the cannulation of the first bone anchor, then diagonally to a proximal opening of the bone tunnel, then distally through the bone tunnel, then diagonally to the proximal head of the locking element, and then distally through the proximal opening of the locking element, the transverse openings of the locking element, and the cannulation of the bone anchor.

Embodiment 15 is the bone fracture repair system of any of embodiments 11 though 14, wherein the shank of the bone anchor has an outer surface comprising a threaded portion and a non-threaded portion.

Embodiment 16 is the bone fracture repair system of any of embodiments 11 though 15, wherein the locking element further includes at least one smooth curved surface bordering the docking slot.

Embodiment 17 is the bone fracture repair system of any of embodiments 11 though 16, wherein docking slot is an open docking slot.

Embodiment 18 is the bone fracture repair system of any of embodiments 11 though 17, wherein the docking slot has a hook configuration

Embodiment 19 is the bone fracture repair system of any of embodiments 11 though 8, wherein the docking slot extends to the distal end of the elongated extension.

Embodiment 20 is the bone fracture repair system of any of embodiments 11 though 19, wherein the elongated extension is sized and configured to snugly nest within the cannulation of the bone anchor to prevent pivoting movement of the locking element when the elongated extension is positioned within the cannulation of the bone anchor.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a front plan view of an example of a bone fracture repair system of the present disclosure, illustrating in particular a first configuration including a pair of bone anchors, a single locking element, and a single tension member, according to some embodiments;

FIG. 2 is a front plan sectional view of the bone fracture repair system of FIG. 1, according to some embodiments;

FIG. 3 is a front plan view of an example of a bone anchor forming part of the bone fracture repair system of FIG. 1, according to some embodiments;

FIG. 4 is a front plan sectional view of the bone anchor of FIG. 3, according to some embodiments;

FIG. 5 is a perspective view of the bone anchor of FIG. 3, according to some embodiments;

FIG. 6 is a top plan view of the bone anchor of FIG. 4, according to some embodiments;

FIGS. 7-8 are perspective views of an example of a locking element forming part of the bone fracture repair system of FIG. 1, according to some embodiments;

FIG. 9 is a side plan view of the locking element of FIG. 7, according to some embodiments;

FIG. 10 is a front plan view of the locking element of FIG. 7, according to some embodiments;

FIG. 11 is a top plan view of the locking element of FIG. 7, according to some embodiments;

FIG. 12 is a flowchart depicting an example method of using the bone fracture repair system of FIG. 1, according to some embodiments;

FIGS. 13-21 illustrate several steps in the method of using the bone fracture repair system of FIG. 1 depicted in the flowchart of FIG. 12, according to some embodiments;

FIG. 22 is a front plan view of the bone fracture repair system of FIG. 1, illustrating in particular a second configuration including a pair of bone anchors, a pair of locking elements, and a pair of tension members, according to some embodiments;

FIG. 23 is a front sectional view of the bone fracture repair system of FIG. 22, according to some embodiments;

FIG. 24 is a flowchart depicting an example method of using the bone fracture repair system of FIG. 22, according to some embodiments;

FIGS. 25-32 illustrate several steps in the method of using the bone fracture repair system of FIG. 1 depicted in the flowchart of FIG. 24, according to some embodiments;

FIG. 33 is a front plan view of another example of a bone fracture repair system, illustrating in particular a first configuration including a pair of bone anchors, a single locking element, and a single tension member, according to some embodiments;

FIG. 34 is a front plan sectional view of the bone fracture repair system of FIG. 12, according to some embodiments;

FIG. 35 is a front plan view of an example of a bone anchor forming part of the bone fracture repair system of FIG. 33, according to some embodiments;

FIG. 36 is a front plan sectional view of the bone anchor of FIG. 35, according to some embodiments;

FIG. 37 is a top plan view of the bone anchor of FIG. 35, according to some embodiments;

FIGS. 38-39 are perspective views of an example of a locking element forming part of the bone fracture repair system of FIG. 33, according to some embodiments;

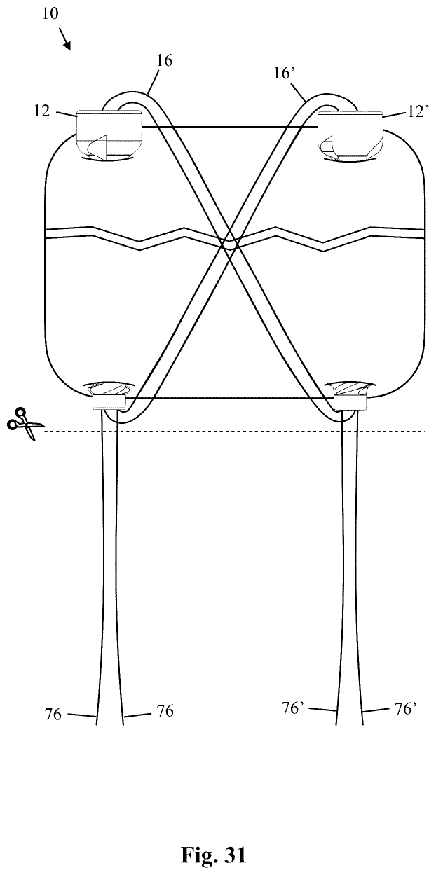

FIG. 40 is a side plan view of the locking element of FIG. 38, according to some embodiments;

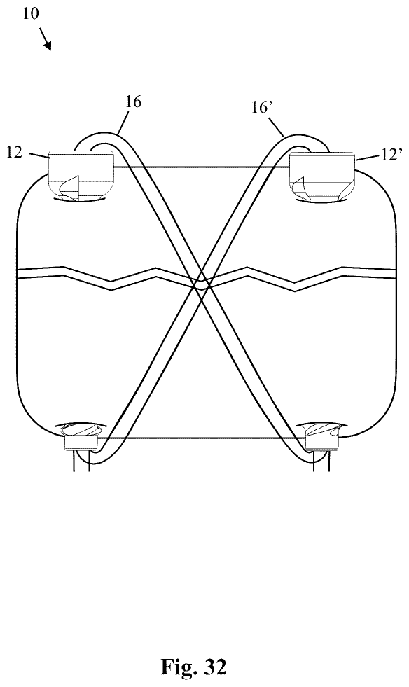

FIG. 41 is a front plan view of the locking element of FIG. 38, according to some embodiments;

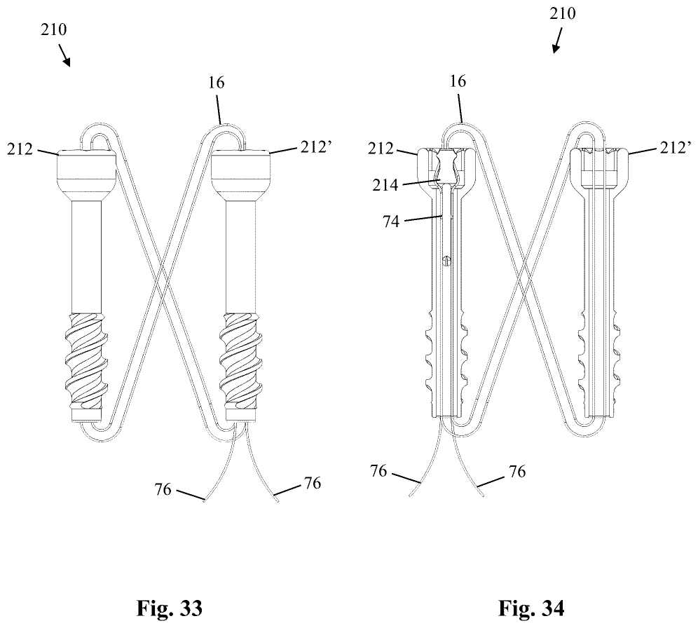

FIG. 42 is a top plan view of the locking element of FIG. 38, according to some embodiments;

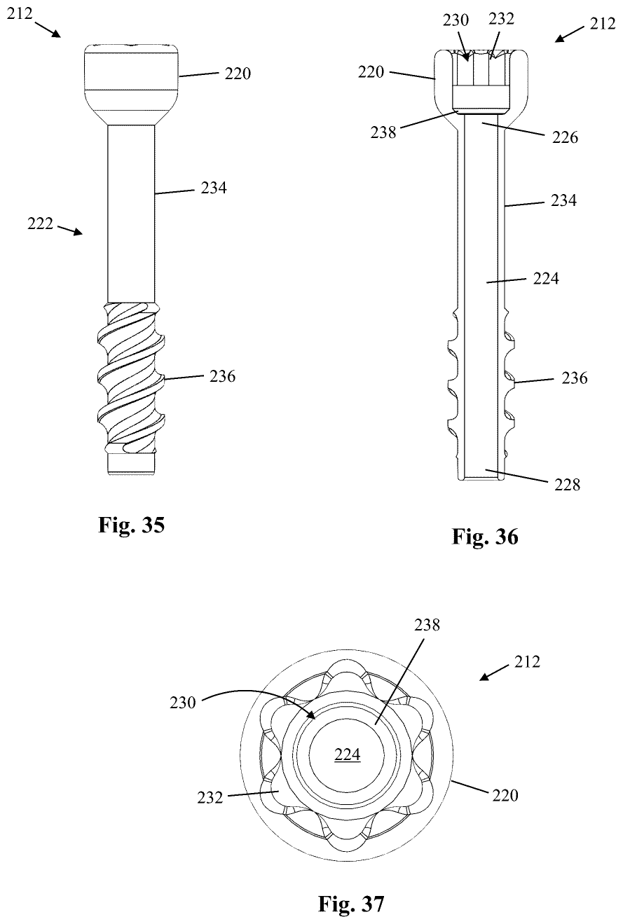

FIG. 43 is a flowchart depicting an example method of using the bone fracture repair system of FIG. 33, according to some embodiments;

FIG. 44 is a perspective sectional view of the bone fracture repair system of FIG. 33, according to some embodiments;

FIG. 45 is a front plan view of the bone fracture repair system of FIG. 33, illustrating in particular a second configuration including a pair of bone anchors, a pair of locking elements, and a pair of tension members, according to some embodiments;

FIG. 46 is a front plan sectional view of the bone fracture repair system of FIG. 45, according to some embodiments;

FIG. 47 is a flowchart depicting an example method of using the bone fracture repair system of FIG. 45, according to some embodiments;

FIG. 48 is a perspective sectional view of the bone fracture repair system of FIG. 45, according to some embodiments;

FIG. 49 is a front plan view of another example of a bone fracture repair system, according to some embodiments;

FIG. 50 is a front plan sectional view of the bone fracture repair system of FIG. 49, according to some embodiments;

FIG. 51 is a front plan sectional view of the bone fracture repair system of FIG. 49 shown in user to repair a bone fracture, according to some embodiments;

FIG. 52 is a perspective sectional view of the bone fracture repair system of FIG. 49 shown in use to repair a bone fracture, according to some embodiments;

FIG. 53 is a front plan view of a bone anchor forming part of the bone fracture repair system of FIG. 49, according to some embodiments;

FIG. 54 is a front plan sectional view of the bone anchor of FIG. 53, according to some embodiments;

FIG. 55 is a perspective view of a locking element forming part of the bone fracture repair system of FIG. 49, according to some embodiments;

FIG. 56 is a flowchart depicting an example method of using the bone fracture repair system of FIG. 49, according to some embodiments;

FIG. 57 is a plan view of an example of a tension member forming part of the bone fracture repair system of FIG. 1, according to some embodiments;

FIG. 58 is a plan view of the tension member of FIG. 57, in particular showing an orientation that includes a looped end and a pair of free ends, according to some embodiments;

FIG. 59 is a front plan view of the bone fracture repair system of FIG. 1, illustrating in particular a third configuration including a single bone anchor, locking element, and tension member, according to some embodiments;

FIG. 60 is a front sectional view of the bone fracture repair system of FIG. 60, according to some embodiments;

FIG. 61 is a flowchart depicting an example method of using the bone fracture repair system of FIG. 59, according to some embodiments; and

FIGS. 62-66 illustrate several steps in the method of using the bone fracture repair system of FIG. 1 depicted in the flowchart of FIG. 61, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The bone fracture repair system and related methods disclosed herein boasts a variety of inventive features and components that warrant patent protection, both individually and in combination.

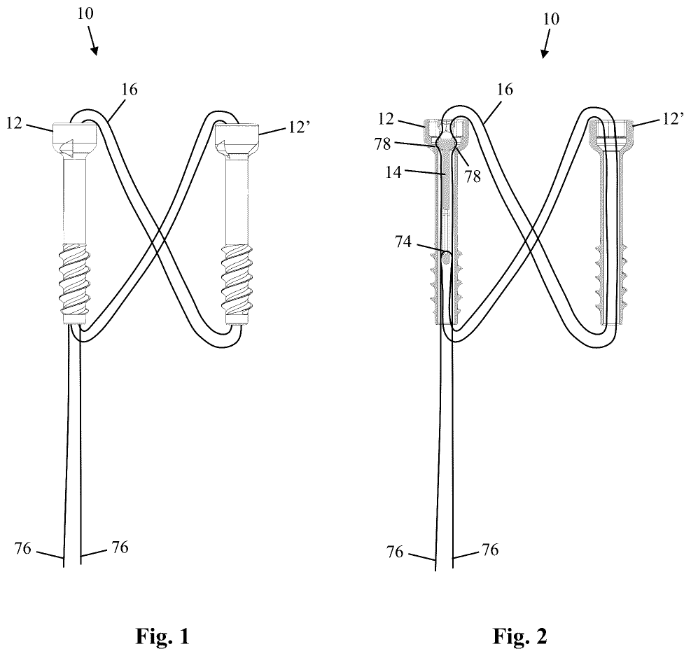

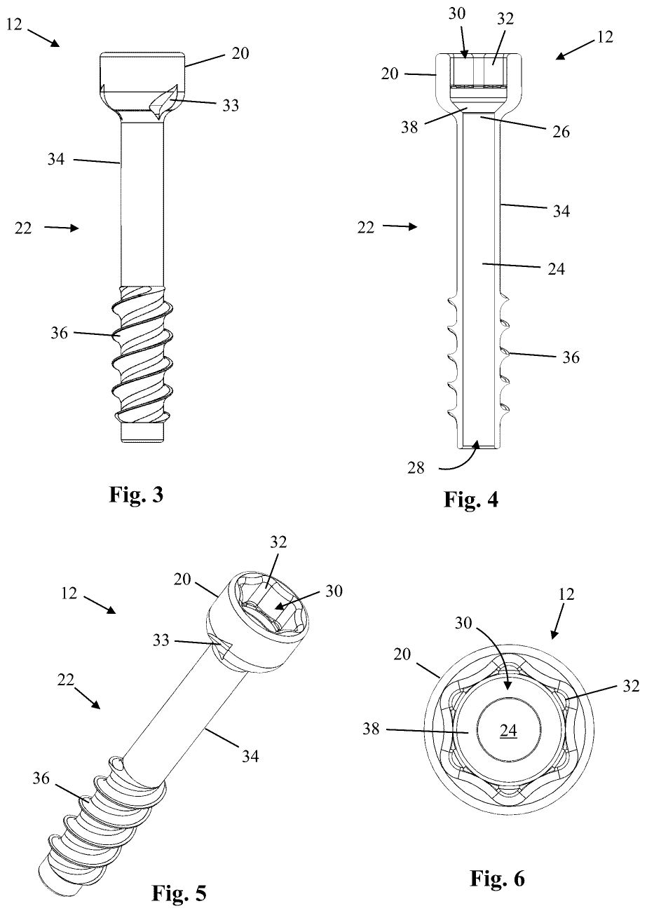

FIGS. 1-2 illustrate one example of a bone fracture repair system 10 using a tension/compression lock configuration, according to some embodiments. Although shown and described herein in relation to a patella fracture, it should be understood that the bone fracture repair system 10 may be used to repair soft tissue and/or fractured bones in any suitable anatomical location in the body, including but not limited to patella, ankle, medial malleolus, olecranon, and the like. In some embodiments, the bone fracture repair system 10 includes at least one bone anchor 12, at least one locking element 14, and at least one flexible tension member 16 configured to interact with the bone anchor 12 and locking element 14 to securely hold fractured bone fragments in place for healing, while also creating a tension band construct that prevents displacement of the bone fragments by forces that tend to pull the bone fragments apart.

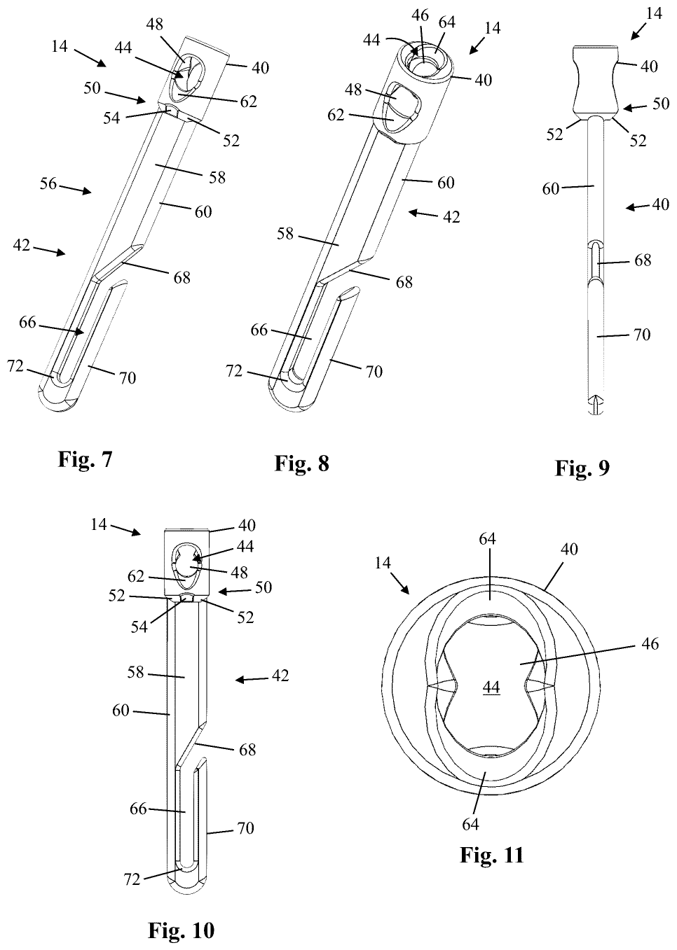

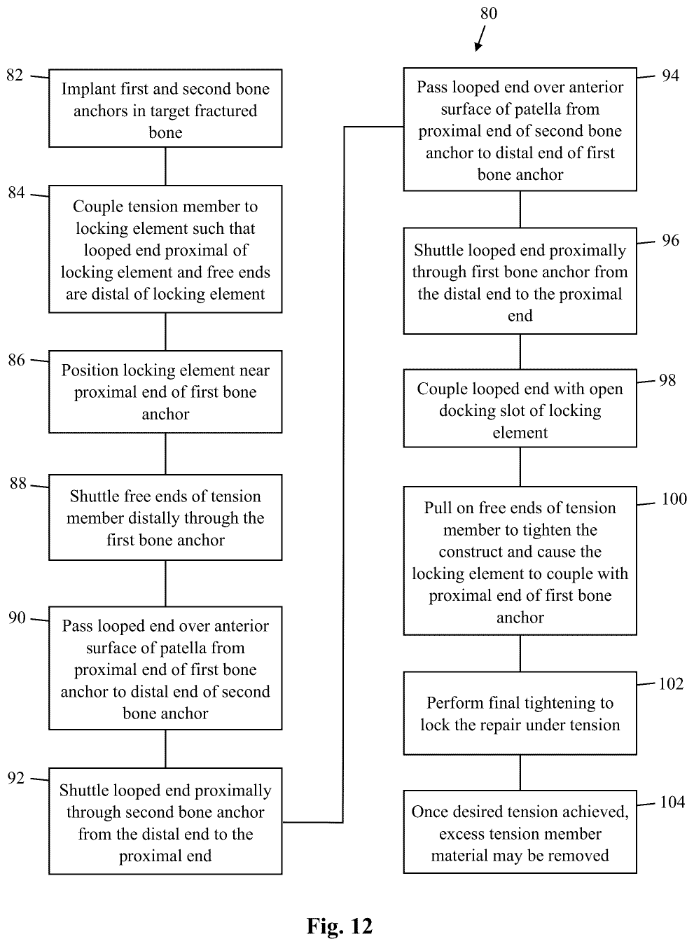

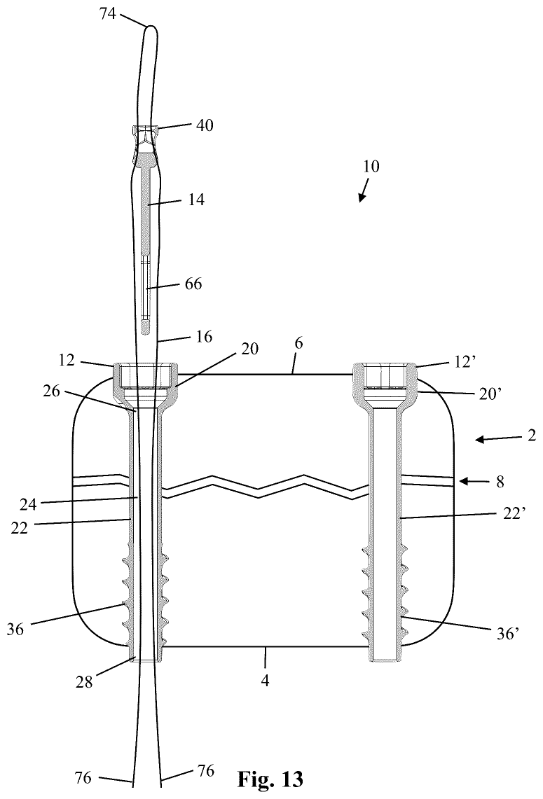

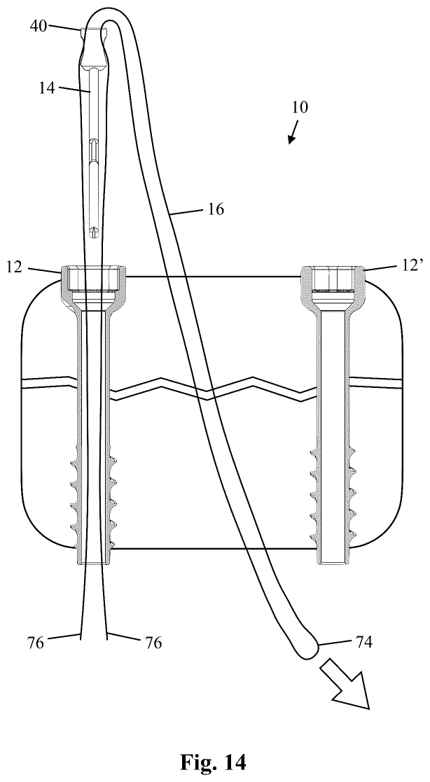

FIGS. 3-6 illustrate an example of a bone anchor 12 forming part of the bone fracture repair system 10, according to some embodiments. In some embodiments, the bone anchor 12 has a head 20 at a proximal end, an elongated shank 22 extending distally from the head 20, and an interior lumen or cannulation 24 extending the length of the shank 22 and having a proximal opening 26 within the head 20 and a distal opening 28 at the distal end of the shank 22. In some embodiments, the head 20 further includes a proximal recess 30 including a drive feature 32 configured to engage with a driver tool. In some embodiments, the head may further include one or more flutes 33 formed in the outer surface to ease insertion of the head into bone. In some embodiments, the shank comprises a cylindrical member having a smooth outer surface portion 34 and a threaded outer surface portion 36. In some embodiments, the threaded outer surface portion 36 is located at or near the distal end of the shank 22, and the smooth outer surface portion 34 is located near the head 20. In some embodiments, the bone anchor 12 may have a fully threaded shank 22. By way of example only, the threaded outer surface portion 36 is configured for insertion into a bone fragment (e.g., the distal bone fragment of the fracture) and gain purchase within the distal bone fragment to hold the distal and proximal bone fragments together. In some embodiments, the head 20 further includes a circumferential mating surface 38 which may be a tapered or concave surface positioned within the proximal recess 30 and surrounding the proximal opening 26 of the shank cannulation 24. In some embodiments, the circumferential mating surface 38 operates as a first locking surface that cooperates with the distal engagement surface 52 and/or a concave cutout 54 positioned within the distal engagement surface 52 of the locking element 14 (e.g. second locking surfaces) to form one or more “pinch points” that capture the tension member 16 therebetween thereby locking the bone fracture repair system 10 under tension. By way of example, the bone anchor 12 may be provided in any suitable length. In some embodiments, the bone anchor 12 may have a shank 22 without a head, and the “pinch point” may be formed at a proximal end of the headless shank 22 for example at a proximal opening of the cannulation 24 or at a location within the cannulation 24.

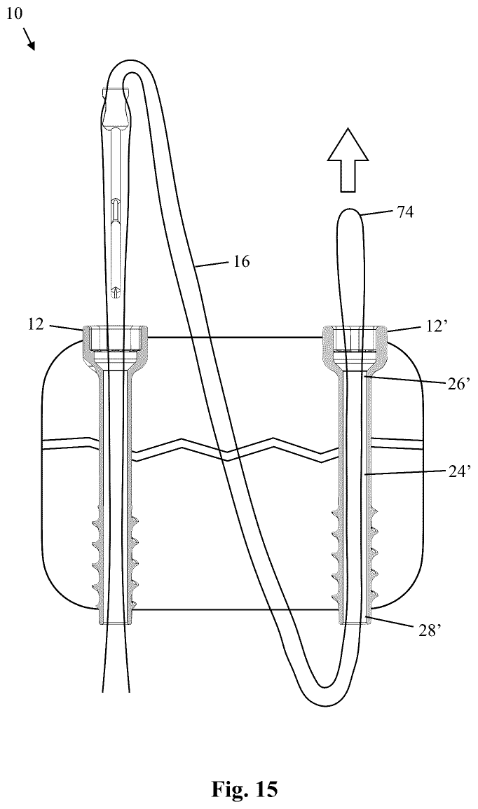

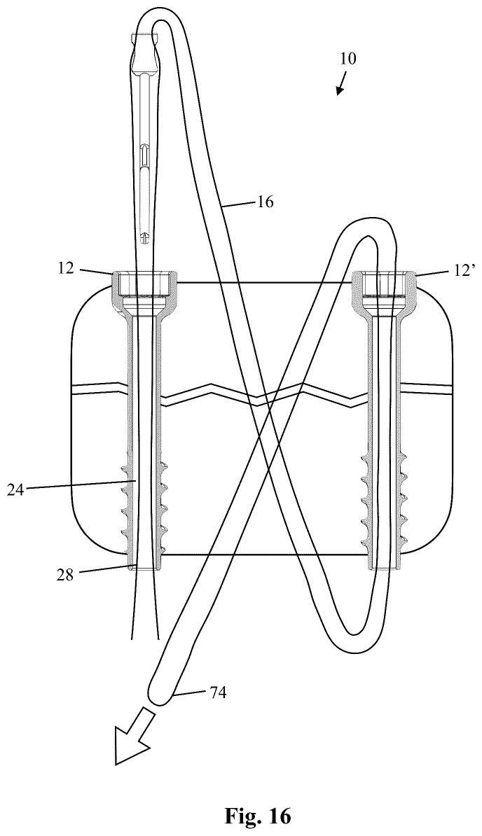

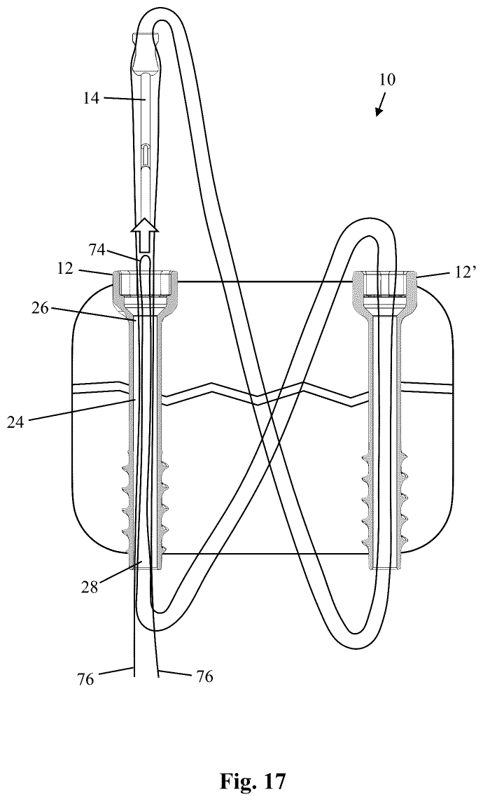

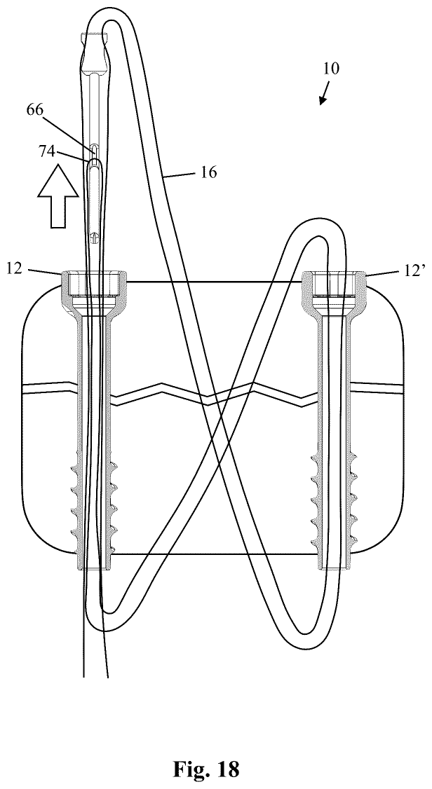

FIGS. 7-11 illustrate an example of a locking element 14 forming part of the bone fracture repair system 10, according to some embodiments. By way of example only, the locking element 14 has a proximal head 40 and a distal extension 42 extending longitudinally from the proximal head 40. In some embodiments, the locking element 14 is configured such that the distal extension 42 is sized and configured to extend into the cannulation 24 of the bone anchor 12 while the head 40 is received within the proximal recess 30.

In some embodiments, the proximal head 40 includes an interior cavity 44 and a plurality of apertures formed in the head 40 that function as ingress and egress ports for the tension member 16 to pass through during assembly and tensioning of the bone fracture repair system 10. For example, in some embodiments the head 40 may have a proximal aperture 46 and a pair of transverse apertures 48 positioned on opposite sides of the head 40. In some embodiments, the proximal head has a bottom or distal portion 50 including a distal engagement surface 52 that may be convex or tapered and may or may not include a concave cutout 54 positioned within the distal engagement surface 52. In some embodiments, the distal engagement surface 52 and/or concave cutout 54 operate as a second locking surface that cooperates with the circumferential mating surface 38 of the bone anchor 12 (e.g., the first locking surface) to form one or more “pinch points” that capture the tension member 16 therebetween thereby locking the bone fracture repair system 10 under tension. By way of example, the concave cutout 54 provides a space for the tension member 16 to pass between the bone anchor 12 and plug insert 14 while still being captured and locked within the concave cutout 54 upon final tightening of the system 10. In some embodiments, the proximal head 40 may include one or more smooth concave lateral recesses 62 positioned near the transverse apertures 48 and that are configured to provide clearance for the tension members 16 to pass during use. In some embodiments, the proximal head 40 may include one or more smooth concave proximal surfaces 64 positioned near the proximal aperture 46 and are configured to provide a smooth contact surface for the tension members 16 as the tension members 16 pass through the head 40 during use.

In some embodiments, the distal extension 42 comprises an elongated body 56 having an oblong shape and including a pair of opposing long sides 58 and a pair of opposing short sides 60. In some embodiments, the short sides 60 are curved and configured to contact or engage with the inner wall of the cannulation 24 of the bone anchor 12 to prevent a toggle motion of the plug insert 14 while engaged with the bone anchor 12. In some embodiments, the long sides 58 are flat or planar and are included to provide space within the cannulation 24 for the tension members 16 to travel through. In some embodiments, the long sides 58 have a convex curvature. In some embodiments, the long sides 58 have a concave curvature.

In some embodiments, the distal extension 42 may include an open docking slot 66 that is open to one of the curved sides 60. In some embodiments, the open docking slot 66 may include an angled proximal surface 68 angling proximally to one of the curved sides 60 (to create the open slot), a hooked end 70, and smooth rounded surfaces 72 bordering the open docking slot 66 on both sides and configured to reduce the effect of friction and/or shearing on the tension members 16 as the tension members 16 are moved within the open docking slot 66 during assembly and/or tightening.

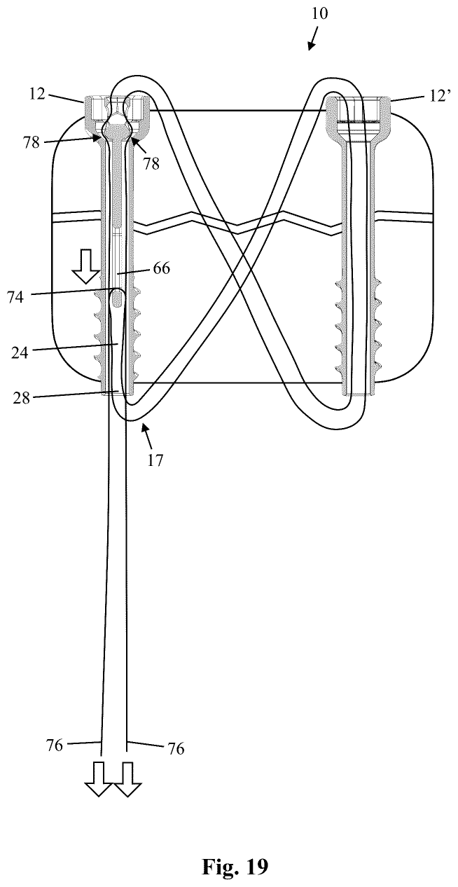

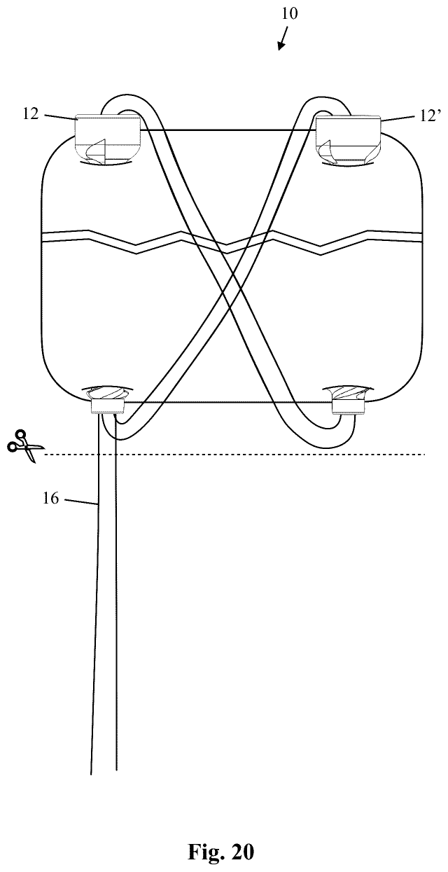

FIG. 12 is a flowchart illustrating various steps of an example method 80 of repairing a bone fracture using the bone fracture repair system 10 described herein. By way of example only, the method 80 is described in the context of repairing a fractured bone 2 (e.g., patella) having a first fracture section 4 and a second fracture section 6 that are separated from one another by a fracture 8, with additional reference to FIGS. 13-21. In this example embodiment, the bone fracture repair system 10 comprises a pair of bone anchors 12, 12′, a single locking element 14, and a single tension member 16. As used throughout this disclosure, the tension member 16 is defined has having a looped portion (or “looped end”) 74 and a pair of free ends 76. By way of example, the looped portion 74 is defined as the portion of the tension member 16 that ultimately engages with (and “loops around”) the docking slot 66 of the locking element 14. The free ends 76 are the actual ends of the tension member 16.

In some embodiments, a first step 82 in the method 80 of repairing a bone fracture is to implant or otherwise position a pair of bone anchors (e.g., first bone anchor 12 and second bone anchor 12′) through a fractured bone (e.g., patella) using a preferred known technique of implanting the bone anchors 12. For example, the bone anchors 12, 12′ may be inserted such that the distal ends of the bone anchors 12, 12′ including the threaded outer surface portions 36, 36′ of the shanks 22, 22′ are seated in the first fracture section 4 and the heads 20, 20′ at the proximal ends of the bone anchors 12, 12′ are positioned within the second fracture section 6, as shown for example in FIG. 13. At this point, the locking element 14 is not coupled to either of the bone anchors 12, 12′.

In some embodiments, a second step 84 in the method 80 of repairing a bone fracture is to couple the tension member 16 to the locking element 14 such that the looped end 74 is positioned proximal of the locking element 14 and the free ends 76 are positioned distal of the locking element 14. By way of example, the locking element 14 and tension member 16 may be coupled by first passing the free ends 76 of the tension member 16 through the proximal aperture 46 of the locking element 14 in a distal direction, and then passing one free end 76 distally through each of the transverse apertures 48 and continuing to advance the free ends 76 distally along the respective long sides 58 of the distal extension 42. This results in the tension member 16 being coupled with the locking element 14 such that the free ends 76 are positioned distally of the locking element 14 and a looped portion 74 is formed proximal of the head 40. In some embodiments, the locking element 14 may be pre-coupled with the tension member 16 for the convenience of the surgeon to minimize the number of steps. In some embodiments, the locking element 14 and the tension member 16 are provided as separate components that are not pre-coupled, and then the user (or assistant, etc.) must shuttle the free ends 76 of the tension member 16 through the locking element 14 as described above. In some embodiments, once the locking element 14 and tension member 16 are coupled (or provided as pre-coupled), a next step 86 of the method 80 of repairing a bone fracture is to position the locking element 14 in the vicinity of the proximal head 20 of the first bone anchor 12.

In some embodiments, a next step 88 in the method 80 of repairing a bone fracture is to shuttle the free ends 76 of the tension member 16 from the proximal end of the bone anchor 12 to the distal end of the bone anchor 12 either by free hand or using a shuttle device (e.g., shuttle nitinol loop or suture loop) to pass the free ends 76 through the proximal opening 26 of the cannulation 24 and out the distal opening 28 of the cannulation 24, as shown by way of example only in FIG. 13.

The user's attention may then turn to the looped portion 74 located proximal of the head 40 of the locking element 14. In some embodiments, a next step 90 of the method 80 of repairing a bone fracture is to pass the looped portion 74 over the anterior surface of the fractured bone 2 from the proximal head 40 of the locking element 14 toward the distal end of the second bone anchor 12′, as shown by way of example in FIG. 14. In some embodiments, a next step 92 of the method 80 of repairing a bone fracture is to shuttle the looped portion 74 proximally through the distal opening 28′ of the cannulation 24′ of the second bone anchor 12′ to the proximal opening 26′ of the cannulation 24′ of the second bone anchor 12′, as shown by way of example in FIG. 15. In some embodiments, once the looped portion 74 exits the proximal opening 26′ of the second bone anchor 12′, a next step 94 of the method 80 of repairing a bone fracture is to pass the looped portion 74 over the anterior surface of the bone (e.g., in a crisscross or “X” pattern) toward the distal opening 28 of the cannulation 24 of the first bone anchor 12, as shown by way of example only in FIG. 16. In some embodiments, a next step 96 of the method 80 of repairing a bone fracture is to shuttle the looped end 74 of the tension member 16 proximally through the cannulation 24 of the first bone anchor 12 from the distal opening 28 through the proximal opening 26 and to the locking element 14 which is located proximal of the proximal opening 26, as shown by way of example in FIG. 17. In some embodiments, a next step 98 of the method 80 of repairing a bone fracture is to couple the looped end 74 of the tension member 16 with the locking element 14 by passing the looped portion 74 into the open docking slot 66 of the locking element 14, as shown by way of example in FIG. 18.

In some embodiments, once this coupling has been established, a next step 100 in the method 80 of repairing a bone fracture is to tighten the system 10 by pulling on the free ends 76 of the tension member 16 that are positioned distal of the distal opening 28 of the cannulation 24 of the first bone anchor 12. Alternatively or additionally, the system 10 can be tightened by pulling on the tension member portion 17, which is part of the portion of tension member 16 exiting from the distal opening 28 and spanning across the bone to the proximal end of the second bone anchor 12′. This allows for seamless translation of the locking element 14 while preventing bunching of the tension member 16 within the cannulation 24. This pulling force causes the locking element 14 to translate in a distal direction into the cannulation 24 of the first bone anchor 12. This can also or alternatively be achieved by manually pushing the locking element 14 distally into the first bone anchor 12.

In some embodiments, once the locking element 14 is seated in the bone anchor 12, a next step 102 in the method 80 of repairing a bone fracture is to lock the repair under tension. To accomplish this, first the user may apply a desired amount of tension to the tension member 16 by pulling the free ends 76 exiting from the distal end of the first bone anchor 12 in a distal direction. By way of example, the free ends may be tensioned one at a time or simultaneously. This will remove the slack out of the system 10, drawing the head 40 of the locking element 14 further into the proximal recess 30 of the head 20 of the bone anchor 12, and creating the “pinch points” 78 described above between the distal engagement surface 52 and/or concave cutout 54 of the locking element 14 and the circumferential mating surface 38 of the bone anchor 12 with the tension member 16 captured between, as shown by way of example in FIG. 19. This capturing creates a knotless compression locking of the tension member 16 under tension, thereby transitioning the bone fracture fixation system 10 from an unlocked state to a locked state, where the locked state is maintained via compression and friction applied to the flexible tension member 16 between the mating surface 38 of the bone anchor 12 and the engagement surface 52 of the locking element 14. This will create a tension band construct which can prevent against displacement of the fracture created by forces that tend to pull the fracture fragments apart, because these counterforces cause an increase in tension in the tension member 16 which in turn causes additional pulling of the looped end 74 on the locking element 14 which only tightens the lock. In some embodiments, a next step 104 of the method 80 of repairing a bone fracture is to optionally remove excess tension member material once the desired tension has been applied and final tightening of the system 10 has occurred, as shown by way of example in FIGS. 20-21.

By way of example, the locking is maintained by the tension created in the bone fracture repair system 10 as well as the looped portion 74 of the tension member 16 which is docked into the open docking slot 66 that continues to assist with preventing any separation of the fracture by providing a counter force in the opposite direction. In some embodiments, if the user desires to revise the tension or if revision surgery proves necessary, the locking element 14 can be loosened by passing a counterforce suture (for example) or other unlocking tool through the transverse apertures 48 and pulling the locking element 14 in a proximal direction. In some embodiments, the counterforce suture or other unlocking tool may be preloaded into the locking element 14. In some embodiments, the counterforce suture or other unlocking tool may be guided through the transverse apertures 48 before final tensioning of the system 10.

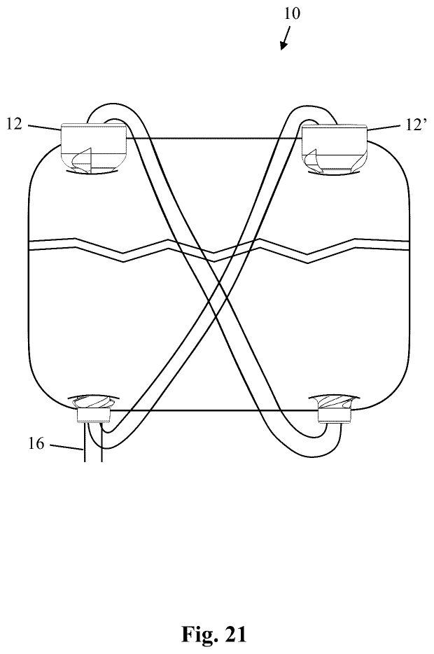

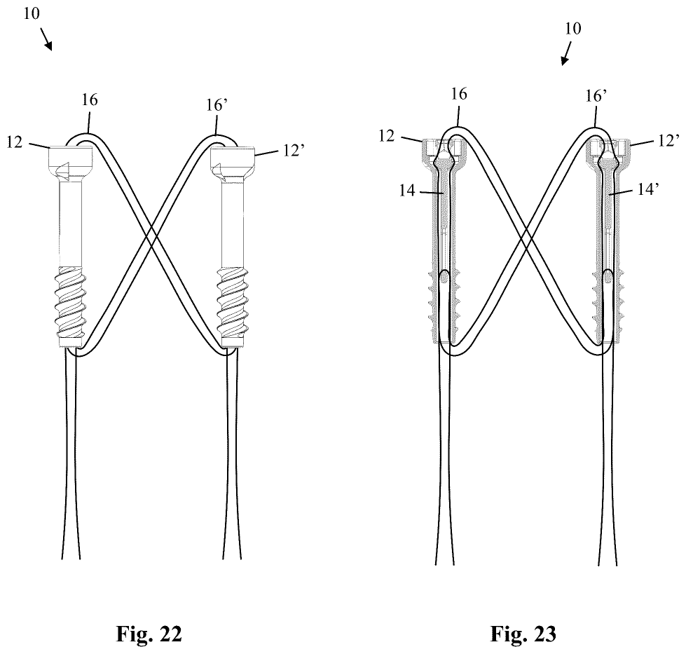

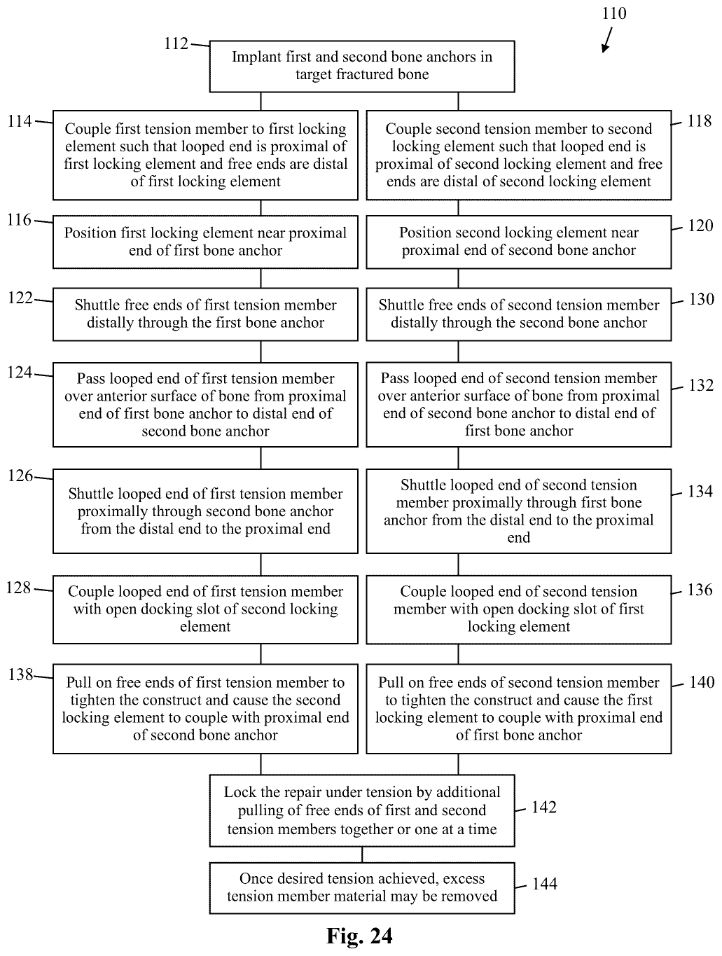

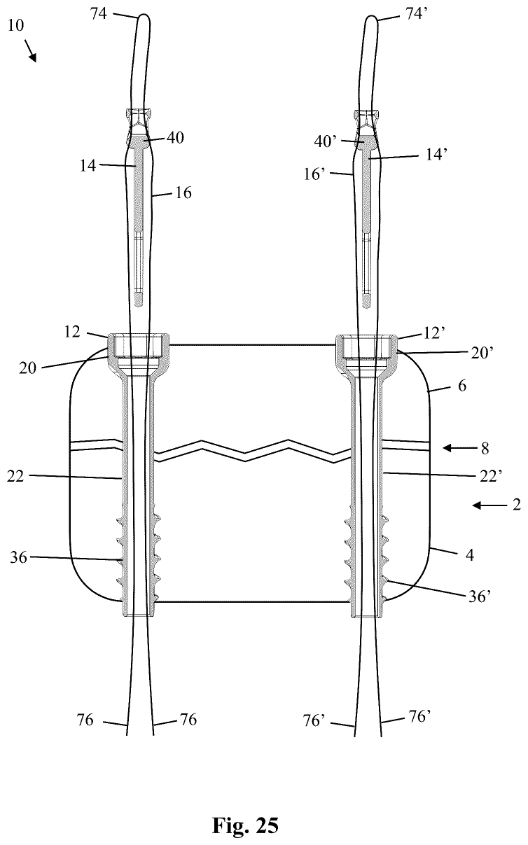

In some embodiments, the bone fracture repair system 10 may achieve a tension band fracture repair using two distinct bone anchors 12 (e.g., a first bone anchor 12 and a second bone anchor 12′), two distinct locking elements 14 (e.g., a first locking element 14 and a second locking element 14′), and two distinct tension members 16 (e.g., a first tension member 16 and a second tension member 16′), as shown by way of example in FIGS. 22-23. FIG. 24 is a flowchart illustrating various steps of an example method 110 of repairing a bone fracture using the bone fracture repair system 10 using two tension members 16, 16′ and two locking elements 14, 14′ as described herein. By way of example, the features of the hardware (e.g., bone anchors 12, 12′, locking elements 14, 14′, and the tension member 16, 16′) are identical to the features of the components as described above. By way of example, the method 110 is described in the context of repairing a fractured bone 2 (e.g., patella) having a first fracture section 4 and a second fracture section 6 that are separated from one another by a fracture 8, with additional reference to FIGS. 25-32.

In some embodiments, a first step 112 in the method 80 of repairing a bone fracture is to implant or otherwise position a pair of bone anchors (e.g., first bone anchor 12 and second bone anchor 12′) through a fractured bone (e.g., patella) using a preferred known technique of implanting the bone anchors 12. For example, the bone anchors 12, 12′ may be inserted such that the distal ends of the bone anchors 12, 12′ including the threaded outer surface portions 36, 36′ of the shanks 22, 22′ are seated in the first fracture section 4 and the heads 20, 20′ at the proximal ends of the bone anchors 12, 12′ are positioned within the second fracture section 6, as shown for example in FIG. 25. At this point, the locking elements 14, 14′ are not coupled to either of the bone anchors 12, 12′.

In some embodiments, a second step 114 in the method 110 of repairing a bone fracture is to couple the first tension member 16 to the first locking element 14 such that the looped end 74 of the first tension member 16 is positioned proximal of the first locking element 14 and the free ends 76 of the first tension member 16 are positioned distal of the first locking element 14. By way of example, the first locking element 14 and first tension member 16 may be coupled by first passing the free ends 76 of the first tension member 16 through the proximal aperture 46 of the first locking element 14 in a distal direction, and then passing one free end 76 distally through each of the transverse apertures 48 and continuing to advance the free ends 76 distally along the respective long sides 58 of the distal extension 42. This results in the first tension member 16 being coupled with the first locking element 14 such that the free ends 76 are positioned distally of the first locking element 14 and a looped portion 74 is formed proximal of the head 40. In some embodiments, the first locking element 14 may be pre-coupled with the first tension member 16 for the convenience of the surgeon to minimize the number of steps. In some embodiments, the first locking element 14 and the first tension member 16 are provided as separate components that are not pre-coupled, and then the user (or assistant, etc.) must shuttle the free ends 76 of the first tension member 16 through the first locking element 14 as described above. In some embodiments, once the first locking element 14 and first tension member 16 are coupled (or provided as pre-coupled), a next step 116 of the method 110 of repairing a bone fracture is to position the first locking element 14 in the vicinity of the proximal head 20 of the first bone anchor 12.

In some embodiments, a next step 118 in the method 110 of repairing a bone fracture is to couple the second tension member 16′ to the second locking element 14′ such that the looped end 74′ of the second tension member 16′ is positioned proximal of the second locking element 14′ and the free ends 76′ of the second tension member 16′ are positioned distal of the second locking element 14′. By way of example, the second locking element 14′ and second tension member 16′ may be coupled by first passing the free ends 76′ of the second tension member 16′ through the proximal aperture 46′ of the second locking element 14′ in a distal direction, and then passing one free end 76′ distally through each of the transverse apertures 48′ and continuing to advance the free ends 76′ distally along the respective long sides 58′ of the distal extension 42′. This results in the second tension member 16′ being coupled with the second locking element 14′ such that the free ends 76′ are positioned distally of the second locking element 14′ and a looped portion 74′ is formed proximal of the head 40′. In some embodiments, the second locking element 14′ may be pre-coupled with the second tension member 16′ for the convenience of the surgeon to minimize the number of steps. In some embodiments, the second locking element 14′ and the second tension member 16′ are provided as separate components that are not pre-coupled, and then the user (or assistant, etc.) must shuttle the free ends 76′ of the second tension member 16′ through the second locking element 14′ as described above. In some embodiments, once the second locking element 14′ and second tension member 16′ are coupled (or provided as pre-coupled), a next step 120 of the method 110 of repairing a bone fracture is to position the second locking element 14′ in the vicinity of the proximal head 20′ of the second bone anchor 12′.

In some embodiments, a next step 122 in the method 110 of repairing a bone fracture is to shuttle the free ends 76 of the first tension member 16 from the proximal end of the first bone anchor 12 to the distal end of the first bone anchor 12 either by free hand or using a shuttle device (e.g., shuttle nitinol loop or suture loop) to pass the free ends 76 through the proximal opening 26 of the cannulation 24 and out the distal opening 28 of the cannulation 24, as shown by way of example only in FIG. 25.

The user's attention may then turn to the looped portion 74 located proximal of the head 40 of the first locking element 14. In some embodiments, a next step 124 of the method 110 of repairing a bone fracture is to pass the looped portion 74 over the anterior surface of the fractured bone 2 from the proximal head 40 of the first locking element 14 toward the distal end of the second bone anchor 12′, as shown by way of example in FIG. 26. In some embodiments, a next step 126 of the method 110 of repairing a bone fracture is to shuttle the looped portion 74 proximally through the distal opening 28′ of the cannulation 24′ of the second bone anchor 12′ to the proximal opening 26′ of the cannulation 24′ of the second bone anchor 12′. In some embodiments, once the looped portion 74 exits the proximal opening 26′ of the second bone anchor 12′, a next step 128 of the method 110 of repairing a bone fracture is to couple the looped end 74 of the first tension member 16 with the second locking element 14′ by passing the looped portion 74 into the open docking slot 66′ of the second locking element 14′, as shown by way of example in FIG. 28.

In some embodiments, a next step 130 in the method 110 of repairing a bone fracture is to shuttle the free ends 76′ of the second tension member 16′ from the proximal end of the second bone anchor 12′ to the distal end of the second bone anchor 12′ either by free hand or using a shuttle device (e.g., shuttle nitinol loop or suture loop) to pass the free ends 76′ through the proximal opening 26′ of the cannulation 24′ and out the distal opening 28′ of the cannulation 24′, as shown by way of example only in FIG. 25.

The user's attention may then turn to the looped portion 74′ located proximal of the head 40′ of the second locking element 14. In some embodiments, a next step 132 of the method 110 of repairing a bone fracture is to pass the looped portion 74′ over the anterior surface of the fractured bone 2 from the proximal head 40′ of the second locking element 14′ toward the distal end of the first bone anchor 12, as shown by way of example in FIG. 27. In some embodiments, a next step 134 of the method 110 of repairing a bone fracture is to shuttle the looped portion 74′ proximally through the distal opening 28 of the cannulation 24 of the first bone anchor 12 to the proximal opening 26 of the cannulation 24 of the first bone anchor 12. In some embodiments, once the looped portion 74′ exits the proximal opening 26 of the first bone anchor 12, a next step 136 of the method 110 of repairing a bone fracture is to couple the looped end 74′ of the second tension member 16′ with the first locking element 14 by passing the looped portion 74′ into the open docking slot 66 of the first locking element 14, as shown by way of example in FIG. 29.

It should be noted that the various steps described herein pertaining to the in situ assembly of the bone fracture repair system 10 can be performed in any reasonable order, for example in some embodiments, the steps described above for coupling the first tension member 16 to the second locking element 14′ may be performed as a group before performing the steps required to for coupling the second tension member 16′ to the first locking element 14. In some embodiments, the various steps may be performed such that the coupling occurs at about the same time (e.g., as shown in FIGS. 25-29) instead of sequentially (as described).

In some embodiments, once these couplings have been established, a next step 138 in the method 110 of repairing a bone fracture is to tighten the system 10 by pulling on the free ends 76 of the first tension member 16 that are positioned distal of the distal opening 28 of the cannulation 24 of the first bone anchor 12. This pulling force causes the second locking element 14′ to translate in a distal direction into the cannulation 24′ of the second bone anchor 12′. Alternatively or additionally, the system 10 can be tightened by pulling on the tension member portion 17, which is part of the portion of the first tension member 16 exiting from the distal opening 28′ of the second bone anchor 12′ and spanning across the bone to the proximal end of the first bone anchor 12. This allows for seamless translation of the second locking element 14′ while preventing bunching of the tension member 16 within the cannulation 24′. This can also or alternatively be achieved by manually pushing the second locking element 14′ distally into the second bone anchor 12′. Similarly, a next step 140 in the method 110 of repairing a bone fracture is to tighten the system 10 by pulling on the free ends 76′ of the second tension member 16′ that are positioned distal of the distal opening 28′ of the cannulation 24′ of the second bone anchor 12′. This pulling force causes the first locking element 14 to translate in a distal direction into the cannulation 24 of the first bone anchor 12. Alternatively or additionally, the system 10 can be tightened by pulling on the tension member portion 17′, which is part of the portion of the second tension member 16′ exiting from the distal opening 28′ and spanning across the bone to the proximal end of the second bone anchor 12′. This allows for seamless translation of the first locking element 14 while preventing bunching of the tension member 16′ within the cannulation 24. This can also or alternatively be achieved by manually pushing the first locking element 14 distally into the first bone anchor 12.

In some embodiments, once the locking elements 14, 14′ are seated in the bone anchors 12, 12′, a next step 142 in the method 110 of repairing a bone fracture is to lock the repair under tension. To accomplish this, first the user may apply a desired amount of tension to the tension members 16, 16′ by pulling the free ends 76, 76′ exiting from the distal end of the bone anchors 12, 12′ in a distal direction. By way of example, the free ends 76, 76′ may be tensioned one at a time or simultaneously. This will remove the slack out of the system 10, drawing the heads 40, 40′ of the locking elements 14, 14′ further into the proximal recesses 30, 30′ of the heads 20, 20′ of the bone anchors 12, 12′, and creating the “pinch points” 78, 78′ described above between the distal engagement surface 52 and/or concave cutout 54 of the locking elements 14, 14′ and the circumferential mating surface 38 of the bone anchors 12, 12′ with the tension members 16, 16′ captured between, as shown by way of example in FIG. 30. This capturing creates a knotless compression locking of the tension members 16, 16′ under tension, thereby transitioning the bone fracture fixation system 10 from an unlocked state to a locked state, where the locked state is maintained via compression and friction applied to the flexible tension members 16, 16′ between the mating surfaces 38 of the bone anchors 12, 12′ and the engagement surfaces 52 of the locking elements 14, 14′. This will create a tension band construct which can prevent against displacement of the fracture created by forces that tend to pull the fracture fragments apart, because these counterforces cause an increase in tension in the tension members 16, 16′ which in turn causes additional pulling of the looped ends 74, 74′ on the locking elements 14, 14′ which only tightens the lock. In some embodiments, a next step 144 of the method 110 of repairing a bone fracture is to optionally remove excess tension member material once the desired tension has been applied and final tightening of the system 10 has occurred, as shown by way of example in FIGS. 31-32.

By way of example, the locking is maintained by the tension created in the bone fracture repair system 10 as well as the looped portions 74, 74′ of the tension members 16, 16′ which are docked into the open docking slots 66, 66′ that continue to assist with preventing any separation of the fracture by providing a counter force in the opposite direction. In some embodiments, if the user desires to revise the tension or if revision surgery proves necessary, the locking elements 14, 14′ can be loosened by passing a counterforce suture (for example) or other unlocking tool through the transverse apertures 48, 48′ and pulling the locking elements 14, 14′ in a proximal direction. In some embodiments, the counterforce suture or other unlocking tool may be preloaded into the locking elements 14, 14′. In some embodiments, the counterforce suture or other unlocking tool may be guided through the transverse apertures 48, 48′ before final tensioning of the system 10.

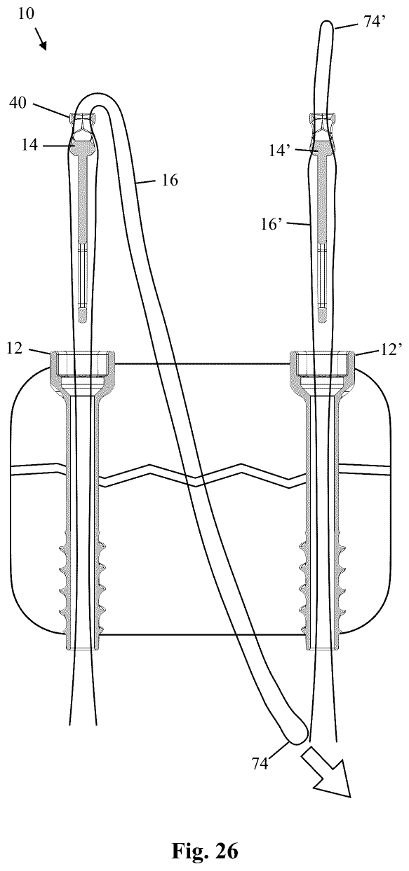

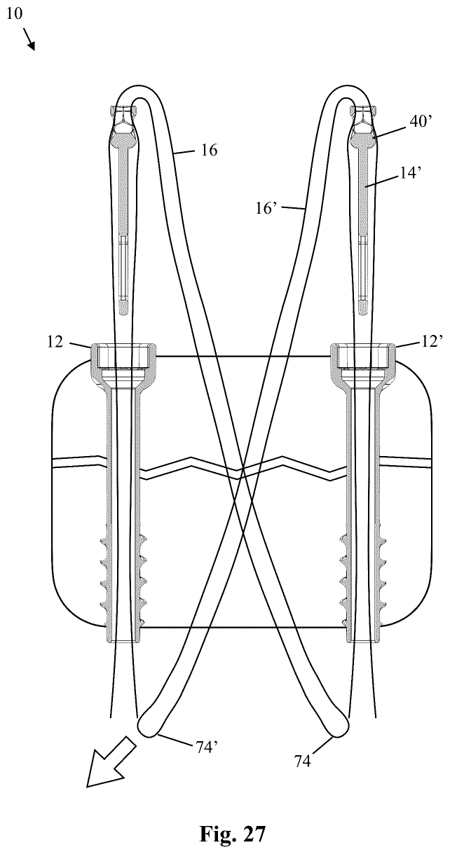

FIGS. 33-34 illustrate another example of a bone fracture repair system 210 using a tension lock configuration, according to some embodiments. Although shown and described herein in relation to a patella fracture, it should be understood that the bone fracture repair system 210 may be used to repair soft tissue and/or fractured bones in any suitable anatomical location in the body, including but not limited to patella, ankle, medial malleolus, olecranon, and the like. In some embodiments, the bone fracture repair system 210 includes at least two bone anchors 212, at least one locking element 214, and a flexible tension member 16 configured to interact with the bone anchors 212 and locking element 214 to securely hold fractured bone fragments in place for healing, while also creating a tension band construct that prevents displacement of the bone fragments by forces that tend to pull the bone fragments apart.

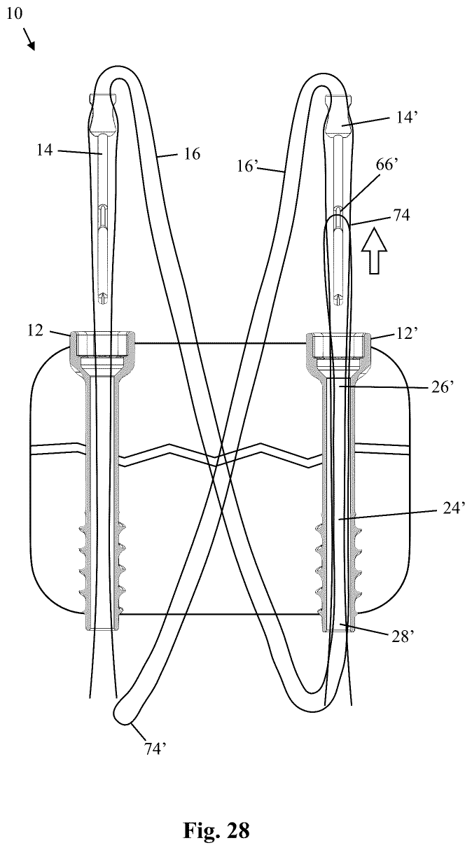

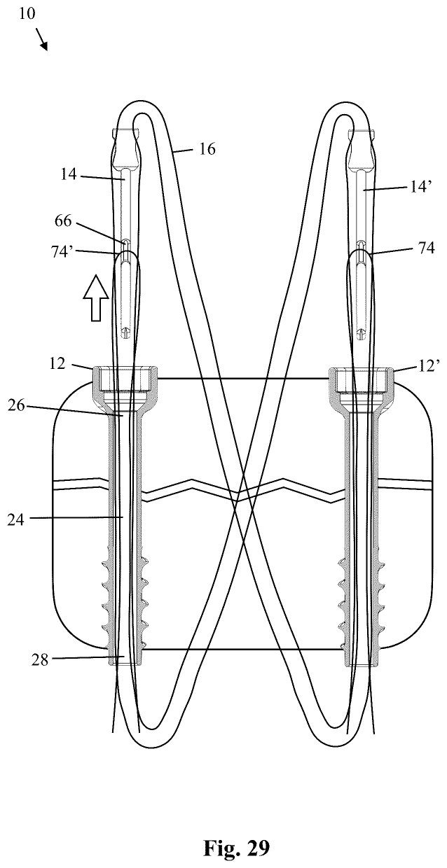

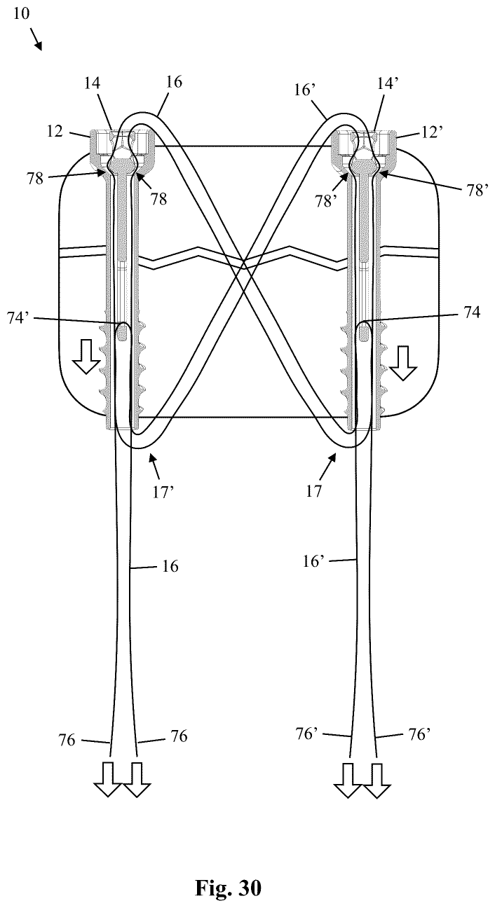

FIGS. 35-37 illustrate an example of a bone anchor 212 forming part of the bone fracture repair system 210, according to some embodiments. In some embodiments, the bone anchor 212 has a head 220 at a proximal end, an elongated shank 222 extending distally from the head 220, and an interior lumen or cannulation 224 extending the length of the shank 222 and having a proximal opening 226 within the head 220 and a distal opening 228 at the distal end of the shank 222. In some embodiments, the head 220 further includes a proximal recess 230 including a drive feature 32 configured to engage with a driver tool. In some embodiments, the shank comprises a cylindrical member having a smooth outer surface portion 234 and a threaded outer surface portion 236. In some embodiments, the threaded outer surface portion 236 is located at or near the distal end of the shank 222, and the smooth outer surface portion 234 is located near the head 220. In some embodiments, the bone anchor 212 may have a fully threaded shank 222. By way of example only, the threaded outer surface portion 236 is configured for insertion into a bone fragment (e.g., the distal bone fragment of the fracture) and gain purchase within the distal bone fragment to hold the distal and proximal bone fragments together. In some embodiments, the head 220 further includes a circumferential mating surface 238 which may be a tapered or concave surface positioned within the proximal recess 30 and surrounding the proximal opening 226 of the shank cannulation 224. In some embodiments, the circumferential mating surface 238 that operates as a “first locking surface” and cooperates with the distal engagement surface 252 and/or a concave cutout 254 (e.g., “second locking surfaces”) positioned within the distal engagement surface 252 of the locking element 214 to form a “pinch point” that captures the tension member 16 therebetween thereby locking the bone fracture repair system 210 under tension. By way of example, the bone anchor 212 may be provided in any suitable length. In some embodiments, the bone anchor 212 may have a shank 222 without a head, and the “pinch point” may be formed at a proximal end of the headless shank 222 for example at a proximal opening of the cannulation 224 or at a location within the cannulation 224.

FIGS. 7-11 illustrate an example of a locking element 214 forming part of the bone fracture repair system 210, according to some embodiments. By way of example only, the locking element 214 has a proximal head 240 and a distal extension 242 extending longitudinally from the proximal head 240. In some embodiments, the locking element 214 is configured such that the distal extension 242 is sized and configured to extend into the cannulation 224 of the bone anchor 212 while the head 240 is received within the proximal recess 230.

In some embodiments, the proximal head 240 includes an interior cavity 244 and a plurality of apertures formed in the head 240 that function as ingress and egress ports for the tension member 16 to pass through during assembly and tensioning of the bone fracture repair system 210. For example, in some embodiments the head 240 may have a proximal aperture 246 and a pair of transverse apertures 248 positioned on opposite sides of the head 240. In some embodiments, the proximal head has a bottom or distal portion 250 including a distal engagement surface 252 that may be convex or tapered and may or may not include a concave cutout 254 positioned within the distal engagement surface 252. In some embodiments, the distal engagement surface 252 and/or concave cutout 254 cooperates with the circumferential mating surface 238 of the bone anchor 212 to form a “pinch point” that captures the tension member 16 therebetween thereby locking the bone fracture repair system 210 under tension. By way of example, the concave cutout 254 provides a space for the tension member 216 to pass between the bone anchor 212 and plug insert 214 while still being captured and locked within the concave cutout 254 upon final tightening of the system 210.

In some embodiments, the distal extension 242 comprises an elongated body 256 having an oblong shape and including a pair of opposing long sides 258 and a pair of opposing short sides 260. In some embodiments, the short sides 260 are curved and configured to contact or engage with the inner wall of the cannulation 224 of the bone anchor 212 to prevent a toggle motion of the plug insert 214 while engaged with the bone anchor 212. In some embodiments, the long sides 258 are flat or planar and are included to provide space within the cannulation 224 for the tension members 16 to travel through. In some embodiments, the long sides 258 have a convex curvature. In some embodiments, the long sides 258 have a concave curvature.

In some embodiments, the distal extension 242 may include one or more docking slots configured to receive the tension member(s) 16 therein. In some embodiments, one or more of the docking slots may comprise a closed docking slot 262 formed as a transverse passage through the long sides 258 of the distal extension 242. In some embodiments, the closed docking slot 262 may have smooth rounded edges 264 to reduce the effect of friction on the tension members 16 as the tension members 16 are moved within the closed docking slot 262 during assembly and/or tightening. In some embodiments, one or more of the docking slots may comprise an open docking slot 266 that is open to one of the curved sides 260. In some embodiments, the open docking slot 266 may include an angled proximal surface 268 angling proximally to one of the curved sides 260 (to create the open slot), a hooked end 270, and smooth rounded surfaces 272 configured to reduce the effect of friction on the tension members 16 as the tension members 16 are moved within the open docking slot 266 during assembly and/or tightening. In some embodiments, the distal extension 242 may include one or more open docking slots and no closed docking slots. In some embodiments, the distal extension 242 may include one or more closed docking slots and no open docking slots.

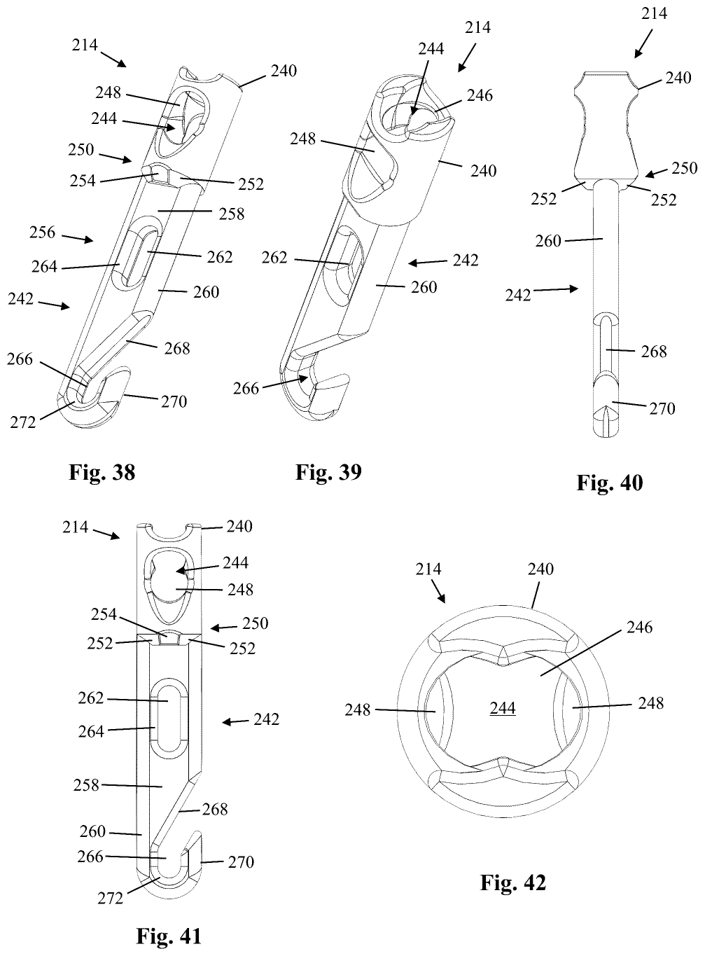

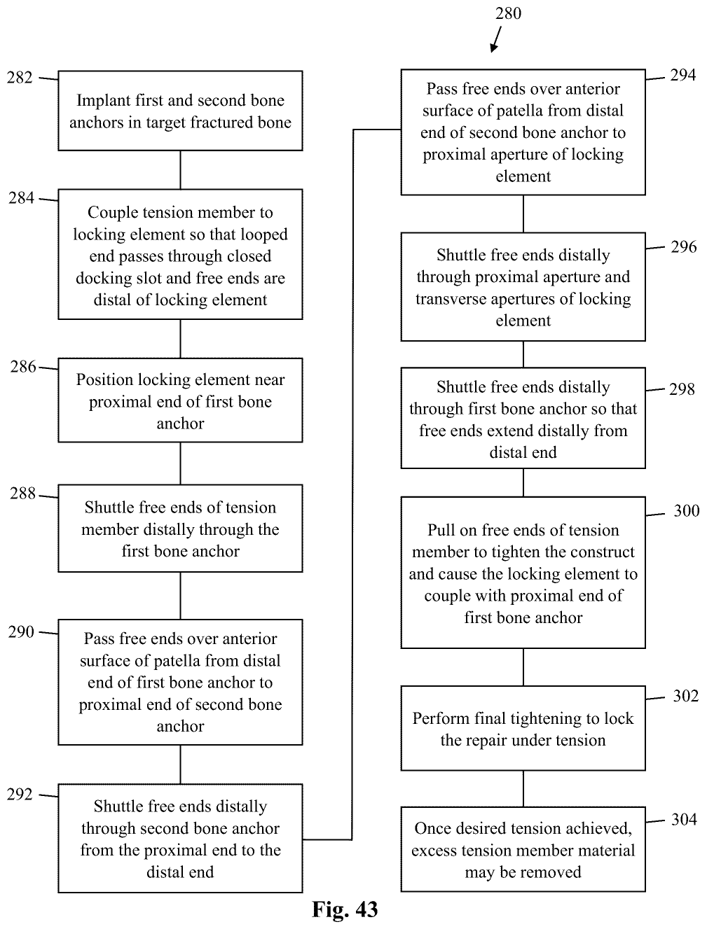

In some embodiments, the same overall assembly may be achieved by chaperoning the free ends 76 of the tension member 16 through the bone anchors 12 instead of the looped end 74 as described above. By way of example, FIG. 43 is a flowchart illustrating various steps of an example method 280 of repairing a bone fracture using the bone fracture repair system 210 described herein. By way of example only, the method 280 is described in the context of repairing a fractured bone 2 (e.g., patella) having a first fracture section 4 and a second fracture section 6 that are separated from one another by a fracture 8, with additional reference to FIG. 44. In this example embodiment, the bone fracture repair system 210 comprises a pair of bone anchors 212, 212′, a single locking element 214, and a single tension member 216.

In some embodiments, a first step in the method 280 of repairing a bone fracture is to implant or otherwise position a pair of bone anchors (e.g., first bone anchor 212 and second bone anchor 212′) through a fractured bone (e.g., patella) using a preferred known technique of implanting the bone anchors 212. For example, the bone anchors 212, 212′ may be inserted such that the distal ends of the bone anchors 212, 212′ including the threaded outer surface portion 236 of the shank 222 is driven into the first fracture section and the head 220 at the proximal end of the bone anchors 212, 212′ is positioned within the second fracture section. At this point, the locking element 214 is not coupled to either of the bone anchors 212, 212′.

In some embodiments, a second step 284 in the method 280 of repairing a bone fracture is to couple the tension member 16 to the locking element 214 such that the looped end 74 is passed through the closed docking slot 262 of the locking element 214 and the free ends 76 are positioned distal of the locking element 214. By way of example, the locking element 214 and tension member 16 may be coupled by first passing the tension member 16 through the closed docking slot 262 and allowing the tension member 16 to settle such that one free end 76 of the tension member 16 is positioned along either long side 258 of the distal extension 242. This results in a coupling in which the free ends 76 are positioned distally of the locking element 214 and the looped portion 74 is positioned within the closed docking slot 262. In some embodiments, the locking element 214 may be pre-coupled with the tension member 16 for the convenience of the surgeon to minimize the number of steps. In some embodiments, the locking element 214 and the tension member 16 are provided as separate components that are not pre-coupled, and then the user (or assistant, etc.) must shuttle the free ends 76 of the tension member 16 through the locking element 214 as described above. In some embodiments, once the locking element 214 and tension member 16 are coupled (or provided as pre-coupled), a next step 288 of the method 280 of repairing a bone fracture is to position the locking element 214 in the vicinity of the proximal head 220 of the first bone anchor 212.

In some embodiments, a next step 288 in the method 280 of repairing a bone fracture is to shuttle the free ends 76 of the tension member 16 from the proximal end of the bone anchor 212 to the distal end of the bone anchor 212 either by free hand or using a shuttle device (e.g., shuttle nitinol loop or suture loop) to pass the free ends 76 through the proximal opening 226 of the cannulation 224 and out the distal opening 228 of the cannulation 224.

In some embodiments, a next step 290 of the method 280 of repairing a bone fracture is to pass the free ends 76 over the anterior surface of the patella from the distal end of the first bone anchor 212 to the proximal end of the second bone anchor 212′. In some embodiments, a next step 292 of the method 280 of repairing a bone fracture is to shuttle the free ends 76 through the proximal opening 226′ of the cannulation 224′ of the second bone anchor 212′ to the distal opening 228′ of the cannulation 224′ of the second bone anchor 212′. In some embodiments, once the free ends 76 exit the distal opening 228′ of the second bone anchor 212′, a next step 294 of the method 280 of repairing a bone fracture is to pass the free ends 76 over the anterior surface of the bone (e.g., in a crisscross or “X” pattern) to the proximal aperture 246 of the locking element 214. In some embodiments, a next step 296 of the method 280 of repairing a bone fracture is to shuttle the free ends 76 of the tension member 16 through the proximal aperture 246 of the locking element 214 in a distal direction, and then passing one free end 76 distally through each of the transverse apertures 248 and continuing to advance the free ends 76 distally along the respective long sides 258 of the distal extension 242. In some embodiments, a next step 298 of the method 280 of repairing a bone fracture is to shuttle the free ends 76 through the cannulation 224 of the first bone anchor 212 from the proximal opening 226 and through the distal opening 228 such that the free ends 76 are positioned distal of the distal opening 228, as shown by way of example in FIG. 44.

In some embodiments, once this assembly has been established, a next step 300 in the method 280 of repairing a bone fracture is to tighten the system 210 by pulling on the free ends 76 of the tension member 16 that are positioned distal of the distal opening 228 of the cannulation 224 of the first bone anchor 212. This pulling force causes the locking element 214 to translate in a distal direction to pull the locking element 214 into the cannulation 224 of the first bone anchor 212. Alternatively or additionally, the system 210 can be tightened by pulling on the tension member portion 17, which is part of the portion of tension member 16 exiting from the distal opening 228 and spanning across the bone to the proximal end of the second bone anchor 212′. This allows for seamless translation of the locking element 214 while preventing bunching of the tension member 16 within the cannulation 224. This can also or alternatively be achieved by manually pushing the locking element 214 distally.

In some embodiments, once the locking element 214 is seated in the bone anchor 212, a next step 302 in the method 380 of repairing a bone fracture is to lock the repair under tension. To accomplish this, first the user may apply a desired amount of tension to the tension member 16 by pulling the free ends 76 and/or the portion of tension member 16 exiting from the distal end of the first bone anchor 212 in a distal direction one at a time (e.g., sequentially) or simultaneously. This will remove the slack out of the system 210, drawing the head 240 of the locking element 214 further into the proximal recess 230 of the head 220 of the bone anchor 212, creating the “pinch points” 78 described above between the distal engagement surface 252 and/or concave cutout 254 of the locking element 214 and the circumferential mating surface 238 of the bone anchor 212 with the tension member 16 captured between. This capturing creates a knotless compression locking of the tension member 16 under tension, thereby transitioning the bone fracture fixation system 210 from an unlocked state to a locked state, where the locked state is maintained via compression and friction applied to the flexible tension member 16 between the mating surface 238 of the bone anchor 212 and the engagement surface 252 of the locking element 214. This will create a tension band construct which can prevent against displacement of the fracture created by forces that tend to pull the fracture fragments apart, because these counterforces cause an increase in tension in the tension member 16 which in turn causes additional pulling of the looped end 74 on the locking element 214 which only tightens the lock. In some embodiments, a next step 304 of the method 380 of repairing a bone fracture is to optionally remove excess tension member material once the desired tension has been applied and final tightening of the system 310 has occurred.

By way of example, locking is maintained by the tension created in the bone fracture repair system 210 as well as the looped portion 74 of the tension member 16 which is docked into the closed docking slot 262 that continues to assist with preventing any separation of the fracture by providing a counter force in the opposite direction. In some embodiments, if the user desires to revise the tension or if revision surgery proves necessary, the locking element 214 can be loosened by passing a counterforce suture (for example) or other unlocking tool through the transverse apertures 248 and pulling the locking element 214 in a proximal direction. In some embodiments, the counterforce suture or other unlocking tool may be preloaded into the locking element 214. In some embodiments, the counterforce suture or other unlocking tool may be guided through the transverse apertures 248 before final tensioning of the system 210.

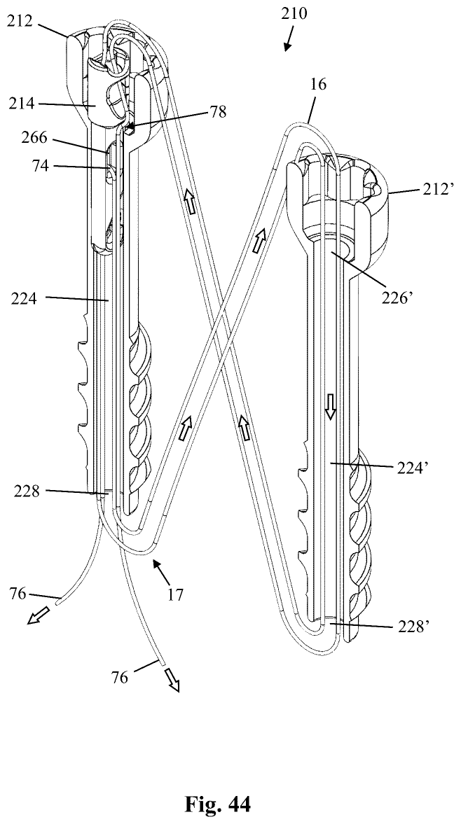

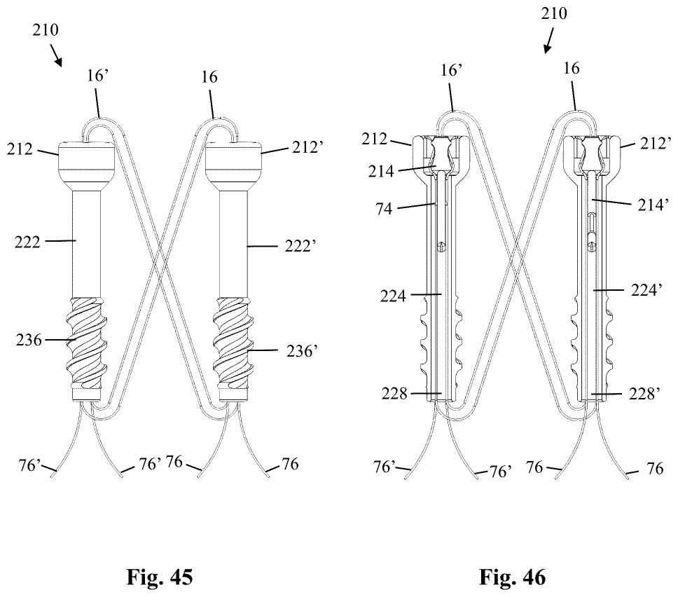

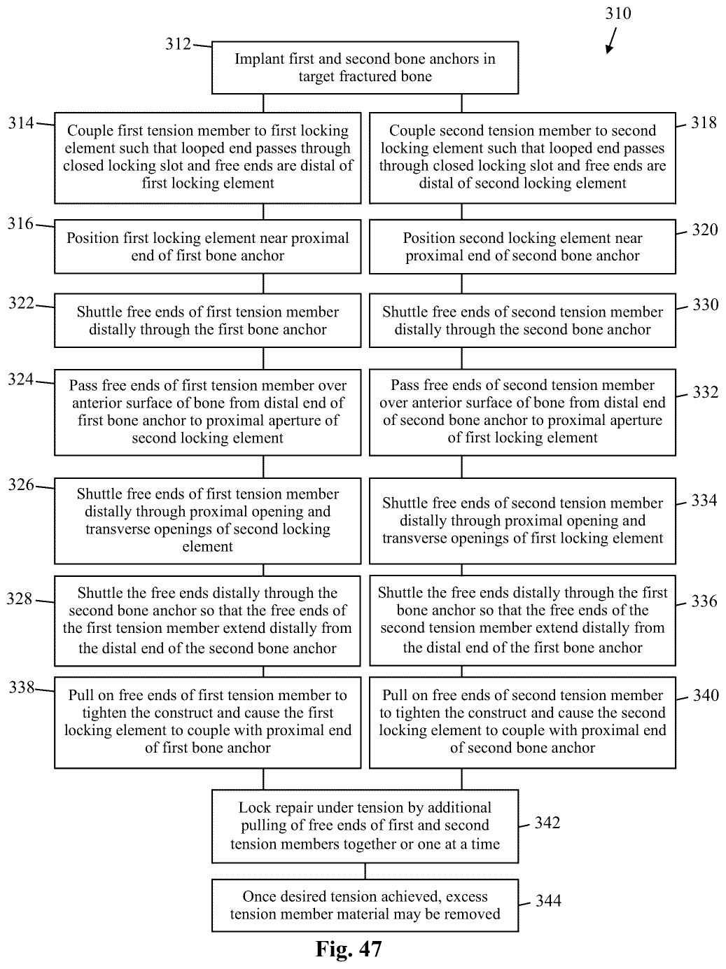

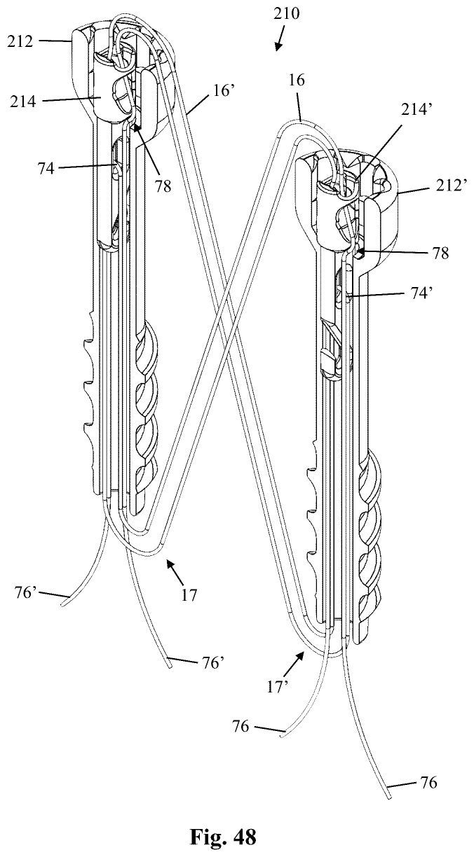

In some embodiments, the bone fracture repair system 210 may achieve a tension band fracture repair using two distinct bone anchors 212 (e.g., a first bone anchor 212 and a second bone anchor 212′), two distinct locking elements 214 (e.g., a first locking element 214 and a second locking element 214′), and two distinct tension members 16 (e.g., a first tension member 16 and a second tension member 16′), as shown by way of example in FIGS. 45-46. FIG. 47 is a flowchart illustrating various steps of an example method 310 of repairing a bone fracture using the bone fracture repair system 210 using two tension members 16, 16′ and two locking elements 214, 214′ as described herein. By way of example, the features of the hardware (e.g., bone anchors 212, 212′, locking elements 214, 214′, and the tension member 16, 16′) are identical to the features of the components as described above. By way of example, the method 310 is described in the context of repairing a fractured bone 2 (e.g., patella) having a first fracture section 4 and a second fracture section 6 that are separated from one another by a fracture 8, with additional reference to FIG. 48.

In some embodiments, a first step 312 in the method 310 of repairing a bone fracture is to implant or otherwise position a pair of bone anchors (e.g., first bone anchor 312 and second bone anchor 312′) through a fractured bone (e.g., patella) using a preferred known technique of implanting the bone anchors 312. For example, the bone anchors 312, 312′ may be inserted such that the distal ends of the bone anchors 312, 312′ including the threaded outer surface portion 336 of the shank 322 are seated in the first fracture section 4 and the heads 320 at the proximal ends of the bone anchors 312, 312′ are positioned within the second fracture section 6. At this point, the locking elements 314, 314′ are not coupled to either of the bone anchors 312, 312′.

In some embodiments, a second step 314 in the method 310 of repairing a bone fracture is to couple the first tension member 16 to the first locking element 214 such that the looped end 74 is passed through the closed docking slot 262 of the first locking element 214 and the free ends 76 are positioned distal of the first locking element 214. By way of example, the first locking element 214 and first tension member 16 may be coupled by first passing the first tension member 16 through the closed docking slot 262 and allowing the first tension member 16 to settle such that one free end 76 of the first tension member 16 is positioned along either long side 258 of the distal extension 242. This results in a coupling in which the free ends 76 are positioned distally of the first locking element 214 and the looped portion 74 is positioned within the closed docking slot 262. In some embodiments, the first locking element 214 may be pre-coupled with the first tension member 16 for the convenience of the surgeon to minimize the number of steps. In some embodiments, the first locking element 214 and the first tension member 16 are provided as separate components that are not pre-coupled, and then the user (or assistant, etc.) must shuttle the free ends 76 of the first tension member 16 through the first locking element 214 as described above. In some embodiments, once the first locking element 214 and first tension member 16 are coupled (or provided as pre-coupled), a next step 316 of the method 310 of repairing a bone fracture is to position the first locking element 214 in the vicinity of the proximal head 220 of the first bone anchor 212.

In some embodiments, a next step 318 in the method 310 of repairing a bone fracture is to couple the second tension member 16′ to the second locking element 214′ such that the looped end 74′ is passed through the closed docking slot 262′ of the second locking element 214′ and the free ends 76′ are positioned distal of the second locking element 214′. By way of example, the second locking element 214′ and second tension member 16′ may be coupled by first passing the second tension member 16′ through the closed docking slot 262′ and allowing the second tension member 16′ to settle such that one free end 76′ of the second tension member 16′ is positioned along either long side 258′ of the distal extension 242′. This results in a coupling in which the free ends 76′ are positioned distally of the second locking element 214′ and the looped portion 74′ is positioned within the closed docking slot 262′. In some embodiments, the second locking element 214′ may be pre-coupled with the second tension member 16′ for the convenience of the surgeon to minimize the number of steps. In some embodiments, the second locking element 214′ and the second tension member 16′ are provided as separate components that are not pre-coupled, and then the user (or assistant, etc.) must shuttle the free ends 76′ of the second tension member 16′ through the second locking element 214′ as described above. In some embodiments, once the second locking element 214′ and second tension member 16′ are coupled (or provided as pre-coupled), a next step 320 of the method 310 of repairing a bone fracture is to position the second locking element 214′ in the vicinity of the proximal head 220′ of the second bone anchor 212′.

In some embodiments, a next step 322 in the method 310 of repairing a bone fracture is to shuttle the free ends 76 of the first tension member 16 from the proximal end of the first bone anchor 212 to the distal end of the first bone anchor 212 either by free hand or using a shuttle device (e.g., shuttle nitinol loop or suture loop) to pass the free ends 76 through the proximal opening 226 of the cannulation 224 and out the distal opening 228 of the cannulation 224. In some embodiments, a next step 324 of the method 310 of repairing a bone fracture is to pass the free ends 76 of the first tension member 16 over the anterior surface of the patella from the distal end of the first bone anchor 212 to the proximal aperture 246′ of the second locking element 214′. In some embodiments, a next step 326 of the method 310 of repairing a bone fracture is to shuttle the free ends 76 of the first tension member 16 through the proximal aperture 246′ of the second locking element 214′ in a distal direction, and then passing one free end 76 distally through each of the transverse apertures 248′ and continuing to advance the free ends 76 distally along the respective long sides 258′ of the distal extension 242′. In some embodiments, a next step 328 of the method 310 of repairing a bone fracture is to shuttle the free ends 76 through the proximal opening 226′ of the cannulation 224′ of the second bone anchor 212′ to the distal opening 228′ of the cannulation 224′ of the second bone anchor 212′such that the free ends 76 of the first tension member 16 are positioned distal of the distal opening 228′ of the second bone anchor 212′, as shown by way of example in FIG. 48.

In some embodiments, a next step 330 in the method 310 of repairing a bone fracture is to shuttle the free ends 76′ of the second tension member 16′ from the proximal end of the second bone anchor 212′ to the distal end of the second bone anchor 212′ either by free hand or using a shuttle device (e.g., shuttle nitinol loop or suture loop) to pass the free ends 76′ through the proximal opening 226′ of the cannulation 224′ and out the distal opening 228′ of the cannulation 224′. In some embodiments, a next step 332 of the method 310 of repairing a bone fracture is to pass the free ends 76′ of the second tension member 16′ over the anterior surface of the patella from the distal end of the second bone anchor 212′ to the proximal aperture 246 of the first locking element 214. In some embodiments, a next step 334 of the method 310 of repairing a bone fracture is to shuttle the free ends 76′ of the second tension member 16′ through the proximal aperture 246 of the first locking element 214 in a distal direction, and then passing one free end 76′ distally through each of the transverse apertures 248 and continuing to advance the free ends 76′ distally along the respective long sides 258 of the distal extension 242. In some embodiments, a next step 336 of the method 310 of repairing a bone fracture is to shuttle the free ends 76′ through the proximal opening 226 of the cannulation 224 of the first bone anchor 212 to the distal opening 228 of the cannulation 224 of the first bone anchor 212 such that the free ends 76′ of the second tension member 16′ are positioned distal of the distal opening 228 of the first bone anchor 212, as shown by way of example in FIG. 48.

It should be noted that the various steps described herein pertaining to the in situ assembly of the bone fracture repair system 210 can be performed in any reasonable order, for example in some embodiments, the steps described above for coupling the first tension member 16 to the second locking element 214′ may be performed as a group before performing the steps required to for coupling the second tension member 16′ to the first locking element 214. In some embodiments, the various steps may be performed such that the coupling occurs at about the same time instead of sequentially.

In some embodiments, once these couplings have been established, a next step 338 in the method 310 of repairing a bone fracture is to tighten the system 210 by pulling on the free ends 76 of the first tension member 16 that are positioned distal of the distal opening 228′ of the cannulation 224′ of the second bone anchor 212′. This pulling force causes the first locking element 214 to translate in a distal direction into the cannulation 224 of the first bone anchor 212. Alternatively or additionally, the system 210 can be tightened by pulling on the tension member portion 17, which is part of the portion of the first tension member 16 exiting from the distal opening 228 of the first bone anchor 212 and spanning across the bone to the proximal end of the second bone anchor 212′. This allows for seamless translation of the first locking element 214 while preventing bunching of the tension member 16 within the cannulation 224. This can also or alternatively be achieved by manually pushing the first locking element 214 distally into the first bone anchor 212. Similarly, a next step 340 in the method 310 of repairing a bone fracture is to tighten the system 210 by pulling on the free ends 76′ of the second tension member 16′ that are positioned distal of the distal opening 228 of the cannulation 224 of the first bone anchor 212. This pulling force causes the second locking element 214′ to translate in a distal direction into the cannulation 224′ of the second bone anchor 212′. Alternatively or additionally, the system 210 can be tightened by pulling on the tension member portion 17′, which is part of the portion of the second tension member 16′ exiting from the distal opening 228′ of the second bone anchor 212′ and spanning across the bone to the proximal end of the first bone anchor 212. This allows for seamless translation of the second locking element 214′ while preventing bunching of the tension member 16 within the cannulation 224′. This can also or alternatively be achieved by manually pushing the second locking element 214 distally into the second bone anchor 212.

In some embodiments, once the locking elements 214, 214′ are seated in the bone anchors 212, 212′, a next step 342 in the method 310 of repairing a bone fracture is to lock the repair under tension. To accomplish this, first the user may apply a desired amount of tension to the tension members 16, 16′ by pulling the free ends 76, 76′ exiting from the distal ends of the bone anchors 212, 212′ in a distal direction. By way of example, the free ends 76, 76′ may be tensioned one at a time or simultaneously. This will remove the slack out of the system 210, drawing the heads 240, 240′ of the locking elements 214, 214′ further into the proximal recesses 230, 230′ of the heads 220, 220′ of the bone anchors 212, 212′, and creating the “pinch points” 78, 78′ described above between the distal engagement surface 252 and/or concave cutout 254 of the locking elements 214, 214′ and the circumferential mating surface 238 of the bone anchors 212, 212′ with the tension members 16, 16′ captured between, as shown by way of example in FIG. 48. This capturing creates a knotless compression locking of the tension members 16, 16′ under tension, thereby transitioning the bone fracture fixation system 210 from an unlocked state to a locked state, where the locked state is maintained via compression and friction applied to the flexible tension members 16, 16′ between the mating surfaces 238 of the bone anchors 212, 212′ and the engagement surfaces 252 of the locking elements 214, 214′. This will create a tension band construct which can prevent against displacement of the fracture created by forces that tend to pull the fracture fragments apart, because these counterforces cause an increase in tension in the tension members 16, 16′ which in turn causes additional pulling of the looped ends 74, 74′ on the locking elements 214, 214′ which only tightens the lock. In some embodiments, a next step 344 of the method 310 of repairing a bone fracture is to optionally remove excess tension member material once the desired tension has been applied and final tightening of the system 310 has occurred.

By way of example, the locking is maintained by the tension created in the bone fracture repair system 210 as well as the looped portions 74, 74′ of the tension members 16, 16′ which are docked into the closed docking slots 262, 262′ that continue to assist with preventing any separation of the fracture by providing a counter force in the opposite direction. In some embodiments, if the user desires to revise the tension or if revision surgery proves necessary, the locking elements 14, 14′ can be loosened by passing a counterforce suture (for example) or other unlocking tool through the transverse apertures 248, 248′ and pulling the locking elements 214, 214′ in a proximal direction. In some embodiments, the counterforce suture or other unlocking tool may be preloaded into the locking elements 214, 214′. In some embodiments, the counterforce suture or other unlocking tool may be guided through the transverse apertures 248, 248′ before final tensioning of the system 210.

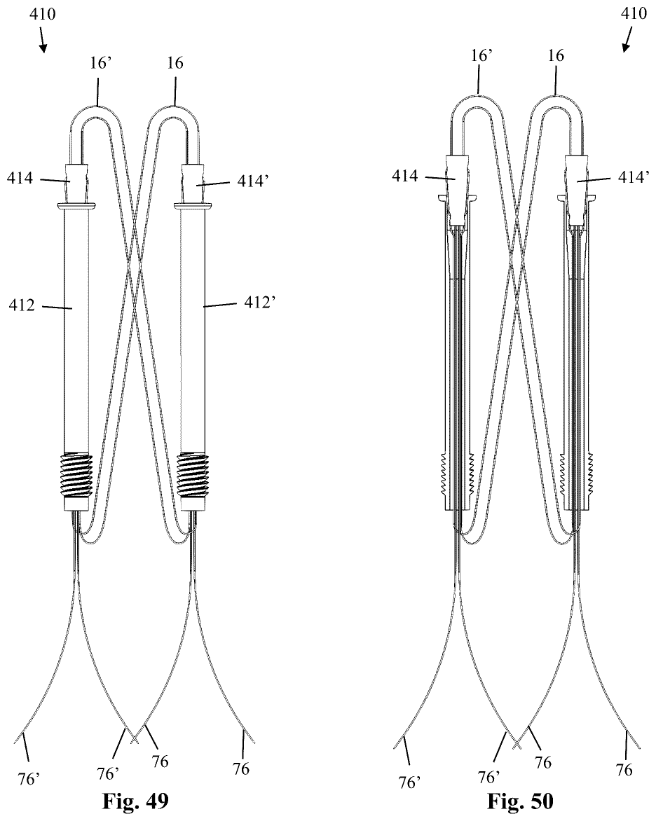

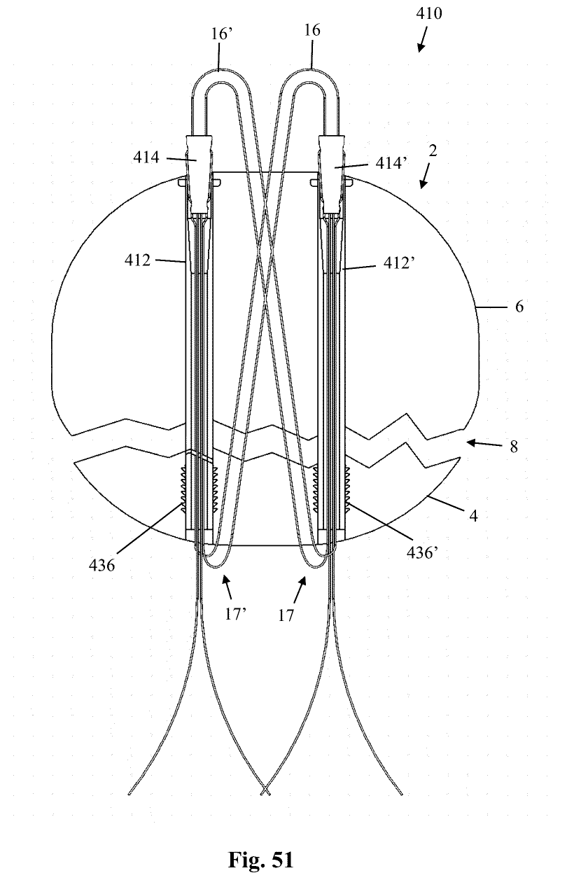

FIGS. 49-55 illustrate one example of a bone fracture repair system 410 using a taper lock configuration, according to some embodiments. Although shown and described herein in relation to a patella fracture, it should be understood that the bone fracture repair system 410 may be used to repair fractured bones in any suitable anatomical location in the body, including but not limited to patella, ankle, and the like. In some embodiments, the bone fracture repair system 410 includes at least two bone anchors 412, 412′, two locking elements 414, 414′, and two flexible tension members 16, 16′ configured to interact with the bone anchors 412, 412′ and locking elements 414, 414′ to securely hold fractured bone fragments in place for healing, while also creating a tension band construct that prevents displacement of the bone fragments by forces that tend to pull the bone fragments apart. In some embodiments, the bone fracture repair system 410 may include only one locking element 414 and flexible tension member 16 in a manner shown and described above in reference to FIGS. 1-2. By way of example, instead of the “pinch points” described above, locking of this embodiment occurs by way of wedging interaction between the locking element 414 and inner surface of bone anchors 412, as will be described below.

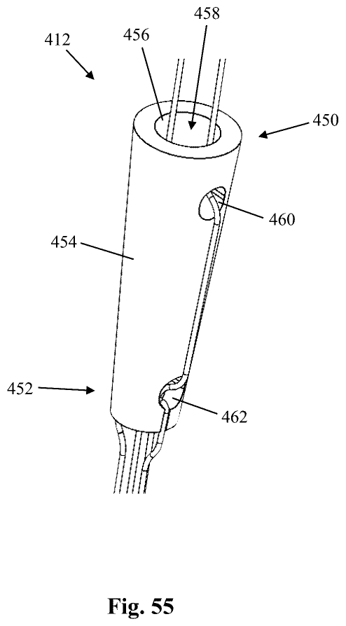

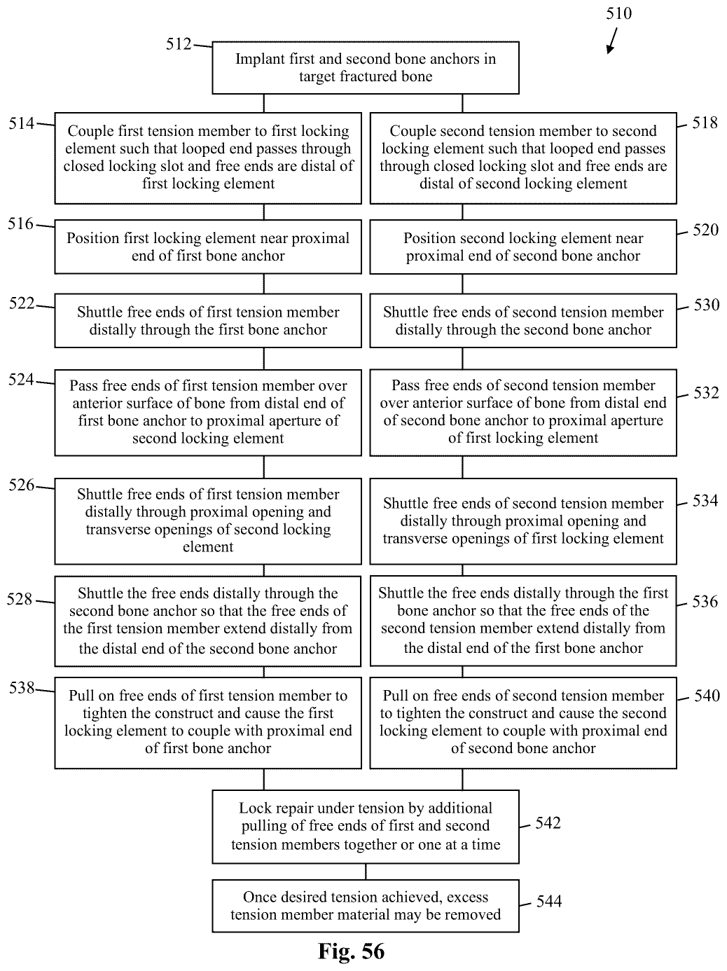

FIGS. 53-54 illustrate an example of a bone anchor 412 (or bone anchor 412′, which is identical to bone anchor 412) forming part of the bone fracture repair system 410, according to some embodiments. By way of example only, the bone anchor 412 has a head 420 at a proximal end, an elongated shank 422 extending distally from the head 420, and an interior lumen or cannulation 424 extending the length of the shank 422 and having a proximal opening 426 within the head 420 and a distal opening 428 at the distal end of the shank 422. In some embodiments, the head 420 further includes a lip 430. In some embodiments, the shank comprises a cylindrical member having a smooth outer surface portion 434 and a threaded outer surface portion 436. In some embodiments, the threaded outer surface portion 436 is located at or near the distal end of the shank 422, and the smooth outer surface portion 434 is located near the head 420. By way of example only, the threaded outer surface portion 436 is configured for insertion into a bone fragment (e.g., the distal bone fragment of the fracture) and gain purchase within the distal bone fragment to hold the distal and proximal bone fragments together. In some embodiments, the bone anchor 412 may have a fully threaded shank 422.

In some embodiments, the cannulation 424 comprises a proximal portion 438 including a cylindrical inner surface 440 having a first diameter, a distal portion 442 including a cylindrical inner surface 444 having a second diameter that is smaller than the first diameter, and a middle portion 446 positioned between the proximal portion 438 and distal portion 442 including a conical inner surface 448 having a diameter that decreases in a proximal to distal direction. In some embodiments, the conical inner surface 448 is sized and configure to interact with the conical outer surface 454 of the locking element 414 to provide an elongated surface area that contacts the tension member 16 to wedge the tension member 16 between the locking element 414 and the conical inner surface 448 of the bone anchor 412 during locking of the bone fracture repair system 410.

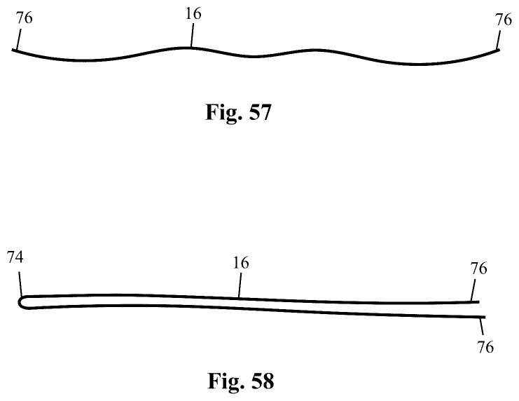

FIG. 55 illustrates an example of a locking element 414 forming part of the bone fracture repair system 410, according to some embodiments. By way of example only, locking element 414 comprises a single body having a proximal portion 450, a distal portion 452, and a conical outer surface 454. In some embodiments, the locking element 414 is configured such that the single body locking element 414 is sized and configured to nest within the middle portion 446 of the cannulation 424 of the bone anchor 412 during tightening of the system 410.

In some embodiments, the proximal portion 450 includes an interior cavity 460 and a plurality of apertures formed in the proximal portion 450 that function as ingress and egress ports for the tension member 416 to pass through during assembly and tensioning of the bone fracture repair system 410. For example, in some embodiments the proximal portion 450 may have a proximal aperture 458 and a pair of transverse apertures 460 positioned on opposite sides of the locking element 414. In some embodiments, distal portion 452 has a docking slot 462 that may be open or closed. In some embodiments, the conical outer surface 454 cooperates with the conical inner surface 448 of the bone anchor 412 to form a locking mechanism that utilizes a combination of tension locking (via the tension member) and interference fit (via the interaction of the conical components described herein) to capture the tension member 16 therebetween thereby locking the construct under tension.

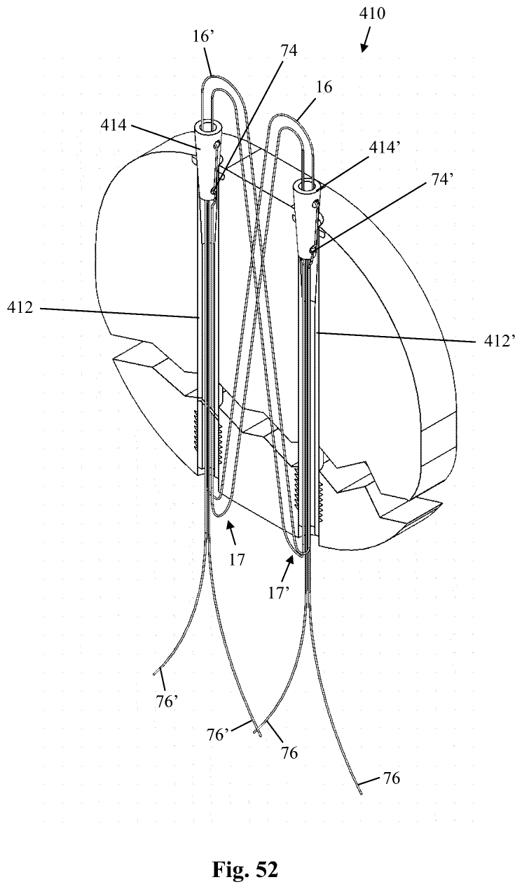

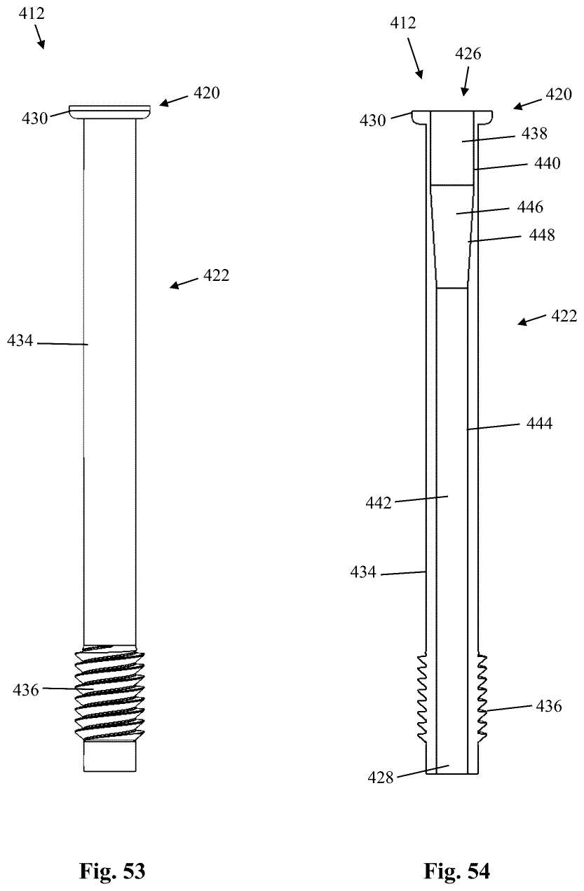

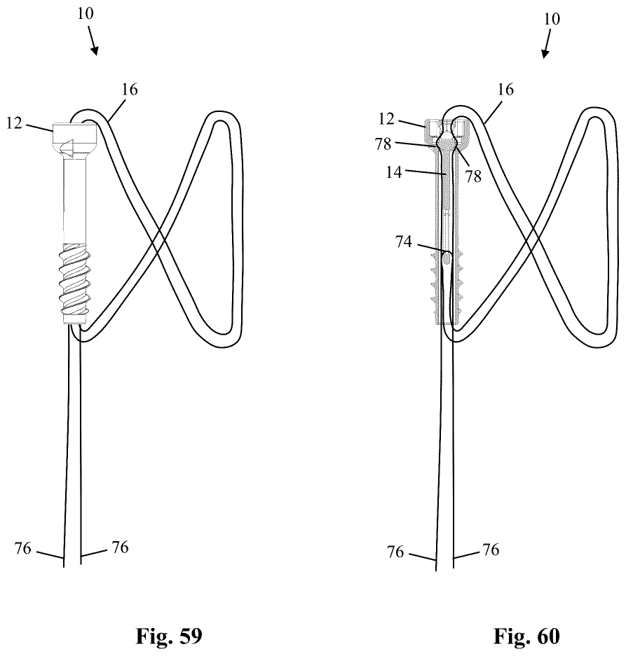

FIG. 56 is a flowchart illustrating various steps of an example method 510 of repairing a bone fracture using the bone fracture repair system 410 using two bone anchors 412, 412′, two tension members 16, 16′ and two locking elements 414, 414′ as described herein. By way of example, the method 510 is described in the context of repairing a fractured bone 2 (e.g., patella) having a first fracture section 4 and a second fracture section 6 that are separated from one another by a fracture 8, with additional reference to FIGS. 51-52. Although described herein as a repair using two locking elements 414, 414′ and two tension members 16, 16′, the bone fracture repair system 410 of the presently described embodiments may be configured for a tension band repair using only one locking element 414 and one tension member 16, with the method steps proceeding as described above with respect to similarly arranged constructs. Moreover, although shown and described herein as having a closed docking slot, in some embodiments, the locking elements 414, 414′ may have open locking slots enabling an assembly technique in which the looped ends of the tension members 16, 16′ are shuttled through the bone anchors 412, 412′ as described above.

In some embodiments, a first step 512 in the method 510 of repairing a bone fracture is to implant or otherwise position a pair of bone anchors (e.g., first bone anchor 512 and second bone anchor 512′) through a fractured bone (e.g., patella) using a preferred known technique of implanting the bone anchors 512. For example, the bone anchors 512, 512′ may be inserted such that the distal ends of the bone anchors 512, 512′ including the threaded outer surface portion 536 of the shank 522 are seated in the first fracture section 4 and the heads 520 at the proximal ends of the bone anchors 512, 512′ are positioned within the second fracture section 6. At this point, the locking elements 514, 514′ are not coupled to either of the bone anchors 512, 512′.

In some embodiments, a second step 514 in the method 510 of repairing a bone fracture is to couple the first tension member 16 to the first locking element 414 such that the looped end 74 is passed through the closed docking slot 462 of the first locking element 414 and the free ends 76 are positioned distal of the first locking element 414. In some embodiments, the first locking element 414 may be pre-coupled with the first tension member 16 for the convenience of the surgeon to minimize the number of steps. In some embodiments, the first locking element 414 and the first tension member 16 are provided as separate components that are not pre-coupled, and then the user (or assistant, etc.) must shuttle the free ends 76 of the first tension member 16 through the first locking element 414 as described above. In some embodiments, once the first locking element 414 and first tension member 16 are coupled (or provided as pre-coupled), a next step 516 of the method 510 of repairing a bone fracture is to position the first locking element 414 in the vicinity of the proximal head 420 of the first bone anchor 412.

In some embodiments, a next step 518 in the method 510 of repairing a bone fracture is to couple the second tension member 16′ to the second locking element 414′ such that the looped end 74′ is passed through the closed docking slot 462′ of the second locking element 414′ and the free ends 76′ are positioned distal of the second locking element 414′. In some embodiments, the second locking element 414′ may be pre-coupled with the second tension member 16′ for the convenience of the surgeon to minimize the number of steps. In some embodiments, the second locking element 414′ and the second tension member 16′ are provided as separate components that are not pre-coupled, and then the user (or assistant, etc.) must shuttle the free ends 76′ of the second tension member 16′ through the second locking element 414′ as described above. In some embodiments, once the second locking element 414′ and second tension member 16′ are coupled (or provided as pre-coupled), a next step 520 of the method 510 of repairing a bone fracture is to position the second locking element 414′ in the vicinity of the proximal head 420′ of the second bone anchor 412′.

In some embodiments, a next step 522 in the method 510 of repairing a bone fracture is to shuttle the free ends 76 of the first tension member 16 from the proximal end of the first bone anchor 412 to the distal end of the first bone anchor 412 either by free hand or using a shuttle device (e.g., shuttle nitinol loop or suture loop) to pass the free ends 76 through the proximal opening 426 of the cannulation 424 and out the distal opening 428 of the cannulation 424. In some embodiments, a next step 524 of the method 510 of repairing a bone fracture is to pass the free ends 76 of the first tension member 16 over the anterior surface of the patella from the distal end of the first bone anchor 412 to the proximal aperture 458′ of the second locking element 414′. In some embodiments, a next step 526 of the method 510 of repairing a bone fracture is to shuttle the free ends 76 of the first tension member 16 through the proximal aperture 458′ of the second locking element 414′ in a distal direction, and then passing one free end 76 distally through each of the transverse apertures 460′ and continuing to advance the free ends 76 distally along the conical outer surface 454′ of the second locking element 414′. In some embodiments, a next step 528 of the method 510 of repairing a bone fracture is to shuttle the free ends 76 through the proximal opening 426′ of the cannulation 424′ of the second bone anchor 412′ to the distal opening 428′ of the cannulation 424′ of the second bone anchor 412′ such that the free ends 76 of the first tension member 16 are positioned distal of the distal opening 428′ of the second bone anchor 412′, as shown by way of example in FIGS. 51-52.

In some embodiments, a next step 530 in the method 510 of repairing a bone fracture is to shuttle the free ends 76′ of the second tension member 16′ from the proximal end of the second bone anchor 412′ to the distal end of the second bone anchor 412′ either by free hand or using a shuttle device (e.g., shuttle nitinol loop or suture loop) to pass the free ends 76′ through the proximal opening 426′ of the cannulation 424′ and out the distal opening 428′ of the cannulation 424′. In some embodiments, a next step 532 of the method 510 of repairing a bone fracture is to pass the free ends 76′ of the second tension member 16′ over the anterior surface of the patella from the distal end of the second bone anchor 412′ to the proximal aperture 458 of the first locking element 414. In some embodiments, a next step 534 of the method 510 of repairing a bone fracture is to shuttle the free ends 76′ of the second tension member 16′ through the proximal aperture 458 of the first locking element 414 in a distal direction, and then passing one free end 76′ distally through each of the transverse apertures 460 and continuing to advance the free ends 76′ distally along the conical outer surface 454 of the first locking element 414. In some embodiments, a next step 536 of the method 510 of repairing a bone fracture is to shuttle the free ends 76′ through the proximal opening 426 of the cannulation 424 of the first bone anchor 412 to the distal opening 428 of the cannulation 424 of the first bone anchor 412 such that the free ends 76′ of the second tension member 16′ are positioned distal of the distal opening 428 of the first bone anchor 412, as shown by way of example in FIGS. 51-52.

It should be noted that the various steps described herein pertaining to the in situ assembly of the bone fracture repair system 510 can be performed in any reasonable order, for example in some embodiments, the steps described above for coupling the first tension member 16 to the second locking element 414′ may be performed as a group before performing the steps required to for coupling the second tension member 16′ to the first locking element 414. In some embodiments, the various steps may be performed such that the coupling occurs at about the same time instead of sequentially.

In some embodiments, once these couplings have been established, a next step 538 in the method 510 of repairing a bone fracture is to tighten the system 510 by pulling on the free ends 76 of the first tension member 16 that are positioned distal of the distal opening 428′ of the cannulation 424′ of the second bone anchor 412′. This pulling force causes the first locking element 414 to translate in a distal direction into the cannulation 424 of the first bone anchor 412. Alternatively or additionally, the system 410 can be tightened by pulling on the tension member portion 17, which is part of the portion of the first tension member 16 exiting from the distal opening 428 of the first bone anchor 412 and spanning across the bone to the proximal end of the second bone anchor 412′. This allows for seamless translation of the first locking element 414 while preventing bunching of the tension member 16 within the cannulation 424. This can also or alternatively be achieved by manually pushing the first locking element 414 distally into the first bone anchor 412. Similarly, a next step 540 in the method 510 of repairing a bone fracture is to tighten the system 410 by pulling on the free ends 76′ of the second tension member 16′ that are positioned distal of the distal opening 428 of the cannulation 424 of the first bone anchor 412. This pulling force causes the second locking element 414′ to translate in a distal direction into the cannulation 424′ of the second bone anchor 412′. Alternatively or additionally, the system 410 can be tightened by pulling on the tension member portion 17′, which is part of the portion of the second tension member 16′ exiting from the distal opening 428′ of the second bone anchor 412′ and spanning across the bone to the proximal end of the first bone anchor 412. This allows for seamless translation of the second locking element 414′ while preventing bunching of the tension member 16 within the cannulation 424′. This can also or alternatively be achieved by manually pushing the second locking element 414′ distally into the second bone anchor 412′.

In some embodiments, once the locking elements 414, 414′ are seated in the bone anchors 212, 212′, a next step 542 in the method 510 of repairing a bone fracture is to lock the repair under tension. To accomplish this, first the user may apply a desired amount of tension to the tension members 16, 16′ by pulling the free ends 76, 76′ exiting from the distal ends of the bone anchors 412, 412′ in a distal direction. By way of example, the free ends 76, 76′ may be tensioned one at a time or simultaneously. This will remove the slack out of the system 210, drawing the first and second locking elements 414, 414′ further into the middle portion 446 of the cannulation 424 of the respective bone anchors 412, 412′, and wedging the tension members 16, 16′ between the conical outer surface 454 of the locking elements 414, 414′ and the conical inner surface 440 of the bone anchors 412, 412′, respectively. This wedging creates a knotless locking of the tension members 16, 16′ under tension, thereby transitioning the bone fracture fixation system 210 from an unlocked state to a locked state, where the locked state is maintained via compression and friction applied to the flexible tension members 16, 16′ between the mating surfaces 238 of the bone anchors 212, 212′ and the engagement surfaces 252 of the locking elements 214, 214′. This will create a tension band construct which can prevent against displacement of the fracture created by forces that tend to pull the fracture fragments apart, because these counterforces cause an increase in tension in the tension members 16, 16′ which in turn causes additional pulling of the looped ends 74, 74′ on the locking elements 414, 414′ which only tightens the lock.

By way of example, the locking is maintained by the tension created in the bone fracture repair system 410 as well as the looped portions 76, 76′ of the tension members 16, 16′ which are docked into the docking slots 462, 462′ that continue to assist with preventing any separation of the fracture by providing a counter force in the opposite direction. In some embodiments, if the user desires to revise the tension or if revision surgery proves necessary, the first and/or second locking elements 414, 414′ can be loosened by passing a counterforce suture (for example) through the transverse apertures 460 and pulling the first and/or second locking elements 414, 414′ in a proximal direction. In some embodiments, the counterforce suture is may be preloaded into the locking elements 414, 414′. In some embodiments, the counterforce suture may be guided through the transverse apertures 460 before final tensioning of the system 410.

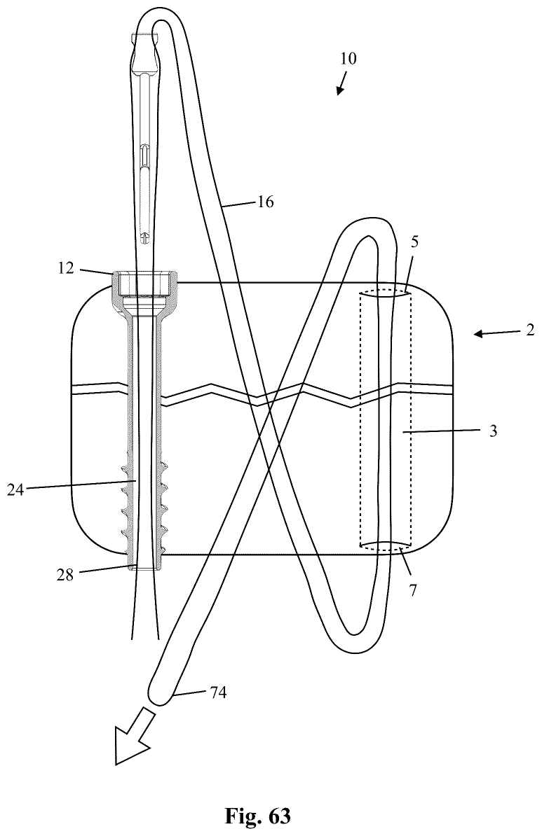

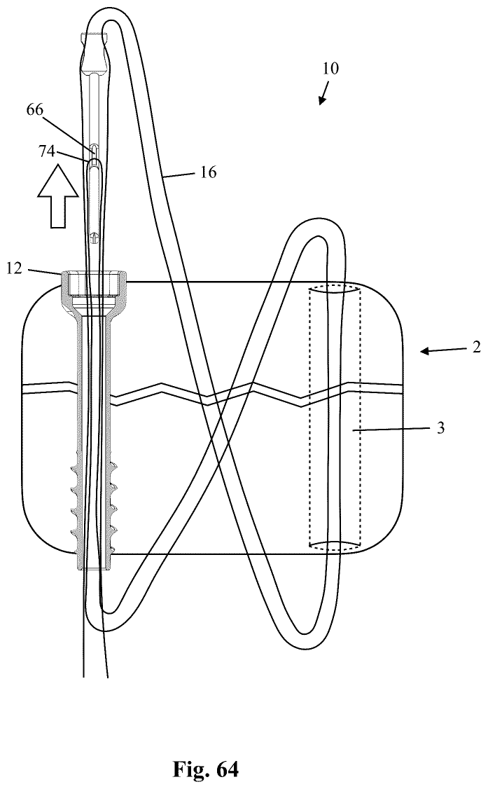

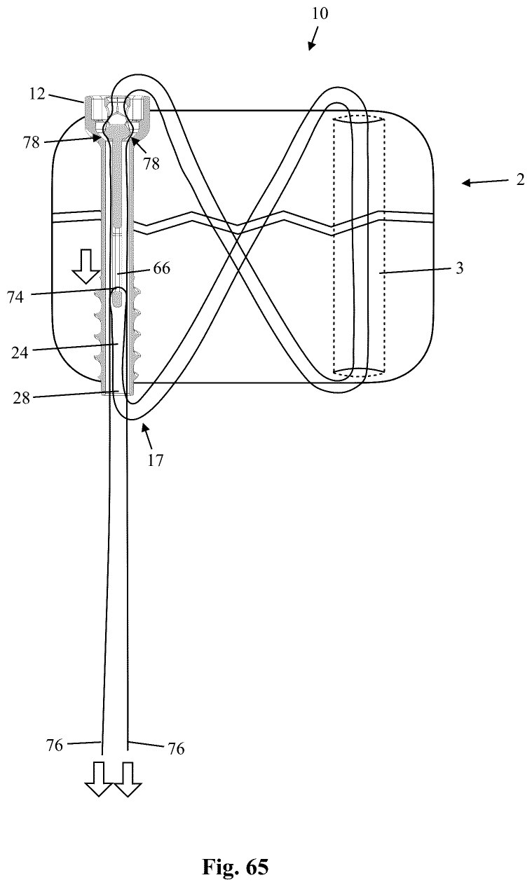

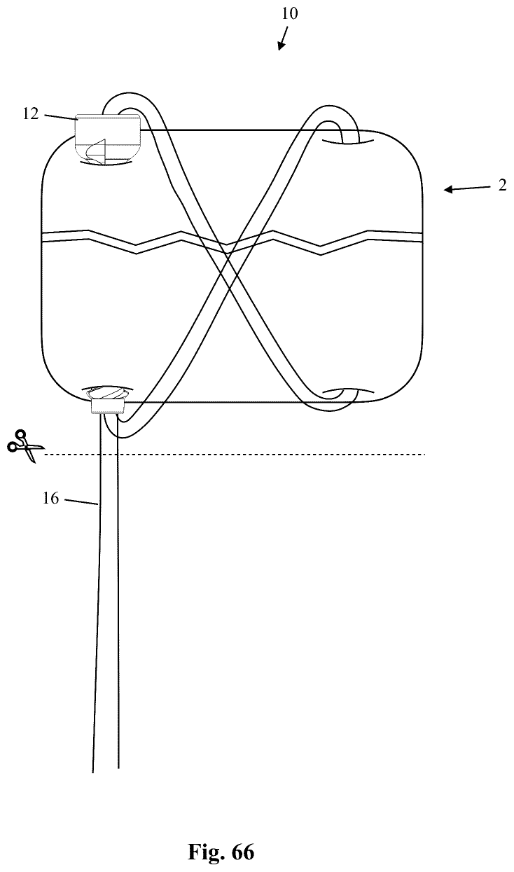

In some embodiments, the bone fracture repair system 10 may achieve a tension band fracture repair using a single bone anchor 12, locking element 14, and tension member 16, as shown by way of example in FIGS. 59-60. FIG. 61 is a flowchart illustrating various steps of an example method 150 of repairing a bone fracture using the bone fracture repair system 10 using a single bone anchor 12, locking element 14, and tension member 16 as described herein. By way of example, the features of the hardware (e.g., bone anchor 12, locking element 14, and the tension member 16) are identical to the features of the components as described above specifically with respect to FIGS. 3-11. By way of example, the method 150 is described in the context of repairing a fractured bone 2 (e.g., patella) having a first fracture section 4 and a second fracture section 6 that are separated from one another by a fracture 8, with additional reference to FIGS. 62-66.

In some embodiments, a first step 152 in the method 150 of repairing a bone fracture is to implant or otherwise position a bone anchor 12 through a fractured bone (e.g., patella) using a preferred known technique of implanting the bone anchor 12. For example, the bone anchor 12 may be inserted such that the distal ends of the bone anchor 12 including the threaded outer surface portion 36 of the shank 22 is seated in the first fracture section 4 and the head 20 at the proximal end of the bone anchors 12 is positioned within the second fracture section 6, as shown for example in FIG. 62. At this point, the locking element 14 is not coupled to the bone anchor 12. Additionally, a bone tunnel 3 is formed within the bone 2 near the bone anchor 12 and extending across the fracture 8, with a distal opening 7 formed in the first bone segment 4 and a proximal opening 5 formed in the second bone segment 6.

In some embodiments, a second step 154 in the method 150 of repairing a bone fracture is to couple the tension member 16 to the locking element 14 such that the looped end 74 is positioned proximal of the locking element 14 and the free ends 76 are positioned distal of the locking element 14. By way of example, the locking element 14 and tension member 16 may be coupled by first passing the free ends 76 of the tension member 16 through the proximal aperture 46 of the locking element 14 in a distal direction, and then passing one free end 76 distally through each of the transverse apertures 48 and continuing to advance the free ends 76 distally along the respective long sides 58 of the distal extension 42. This results in the tension member 16 being coupled with the locking element 14 such that the free ends 76 are positioned distally of the locking element 14 and a looped portion 74 is formed proximal of the head 40. In some embodiments, the locking element 14 may be pre-coupled with the tension member 16 for the convenience of the surgeon to minimize the number of steps. In some embodiments, the locking element 14 and the tension member 16 are provided as separate components that are not pre-coupled, and then the user (or assistant, etc.) must shuttle the free ends 76 of the tension member 16 through the locking element 14 as described above. In some embodiments, once the locking element 14 and tension member 16 are coupled (or provided as pre-coupled), a next step 156 of the method 80 of repairing a bone fracture is to position the locking element 14 in the vicinity of the proximal head 20 of the bone anchor 12.

In some embodiments, a next step 158 in the method 150 of repairing a bone fracture is to shuttle the free ends 76 of the tension member 16 from the proximal end of the bone anchor 12 to the distal end of the bone anchor 12 either by free hand or using a shuttle device (e.g., shuttle nitinol loop or suture loop) to pass the free ends 76 through the proximal opening 26 of the cannulation 24 and out the distal opening 28 of the cannulation 24, as shown by way of example only in FIG. 62.

The user's attention may then turn to the looped portion 74 located proximal of the head 40 of the locking element 14. In some embodiments, a next step 160 of the method 150 of repairing a bone fracture is to pass the looped portion 74 over the anterior surface of the fractured bone 2 from the proximal head 40 of the locking element 14 toward the distal opening 7 of the bone tunnel 3. In some embodiments, a next step 162 of the method 150 of repairing a bone fracture is to shuttle the looped portion 74 proximally through the bone tunnel 3 to the proximal opening 5 of the bone tunnel 3. In some embodiments, once the looped portion 74 exits the proximal opening 5 of the bone tunnel 3, a next step 164 of the method 150 of repairing a bone fracture is to pass the looped portion 74 over the anterior surface of the bone 2 (e.g., in a crisscross or “X” pattern) toward the distal opening 28 of the cannulation 24 of the first bone anchor 12, as shown by way of example only in FIG. 63. In some embodiments, a next step 166 of the method 150 of repairing a bone fracture is to shuttle the looped end 74 of the tension member 16 proximally through the cannulation 24 of the first bone anchor 12 from the distal opening 28 through the proximal opening 26 and to the locking element 14 which is located proximal of the proximal opening 26, as shown by way of example in FIG. 64. In some embodiments, a next step 168 of the method 150 of repairing a bone fracture is to couple the looped end 74 of the tension member 16 with the locking element 14 by passing the looped portion 74 into the open docking slot 66 of the locking element 14, as shown by way of example in FIG. 64.

In some embodiments, once this coupling has been established, a next step 170 in the method 150 of repairing a bone fracture is to tighten the system 10 by pulling on the free ends 76 of the tension member 16 that are positioned distal of the distal opening 28 of the cannulation 24 of the first bone anchor 12. Alternatively or additionally, the system 10 can be tightened by pulling on the tension member portion 17, which is part of the portion of tension member 16 exiting from the distal opening 28 and spanning across the bone to the proximal opening 5 of the bone anchor tunnel 3. This allows for seamless translation of the locking element 14 while preventing bunching of the tension member 16 within the cannulation 24. This pulling force causes the locking element 14 to translate in a distal direction into the cannulation 24 of the first bone anchor 12. This can also or alternatively be achieved by manually pushing the locking element 14 distally into the first bone anchor 12.

In some embodiments, once the locking element 14 is seated in the bone anchor 12, a next step 172 in the method 150 of repairing a bone fracture is to lock the repair under tension. To accomplish this, first the user may apply a desired amount of tension to the tension member 16 by pulling the free ends 76 exiting from the distal end of the first bone anchor 12 in a distal direction. By way of example, the free ends may be tensioned one at a time or simultaneously. This will remove the slack out of the system 10, drawing the head 40 of the locking element 14 further into the proximal recess 30 of the head 20 of the bone anchor 12, and creating the “pinch points” 78 described above between the distal engagement surface 52 and/or concave cutout 54 of the locking element 14 and the circumferential mating surface 38 of the bone anchor 12 with the tension member 16 captured between, as shown by way of example in FIG. 65. This capturing creates a knotless compression locking of the tension member 16 under tension, thereby transitioning the bone fracture fixation system 10 from an unlocked state to a locked state, where the locked state is maintained via compression and friction applied to the flexible tension member 16 between the mating surface 38 of the bone anchor 12 and the engagement surface 52 of the locking element 14. This will create a tension band construct which can prevent against displacement of the fracture created by forces that tend to pull the fracture fragments apart, because these counterforces cause an increase in tension in the tension member 16 which in turn causes additional pulling of the looped end 74 on the locking element 14 which only tightens the lock. In some embodiments, a next step 104 of the method 80 of repairing a bone fracture is to optionally remove excess tension member material once the desired tension has been applied and final tightening of the system 10 has occurred, as shown by way of example in FIG. 66.

By way of example, the locking is maintained by the tension created in the bone fracture repair system 10 as well as the looped portion 74 of the tension member 16 which is docked into the open docking slot 66 that continues to assist with preventing any separation of the fracture by providing a counter force in the opposite direction. In some embodiments, if the user desires to revise the tension or if revision surgery proves necessary, the locking element 14 can be loosened by passing a counterforce suture (for example) or other unlocking tool through the transverse apertures 48 and pulling the locking element 14 in a proximal direction. In some embodiments, the counterforce suture or other unlocking tool may be preloaded into the locking element 14. In some embodiments, the counterforce suture or other unlocking tool may be guided through the transverse apertures 48 before final tensioning of the system 10.

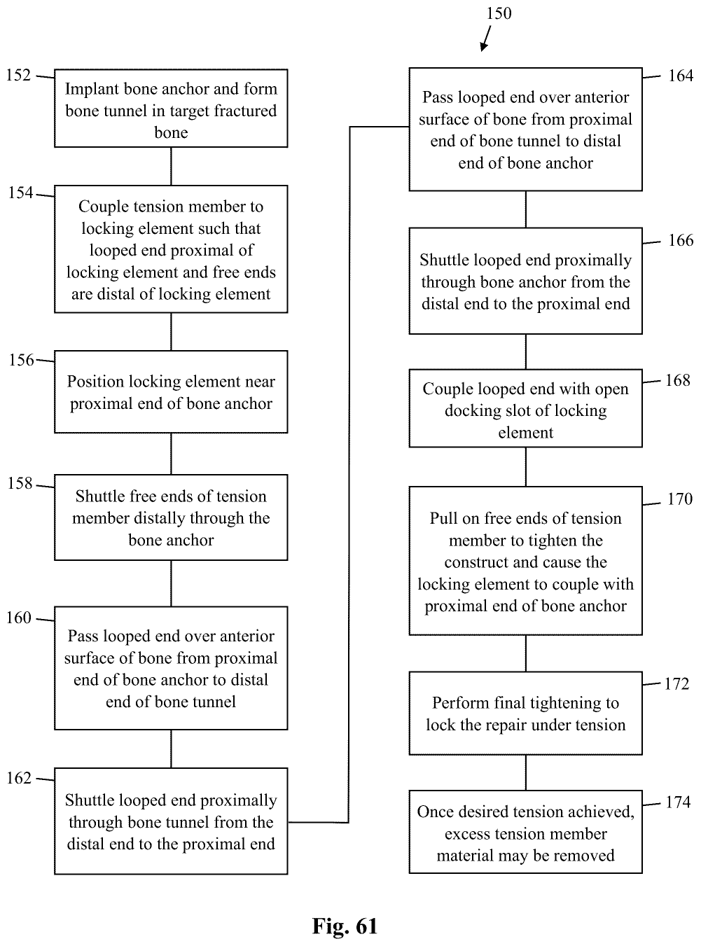

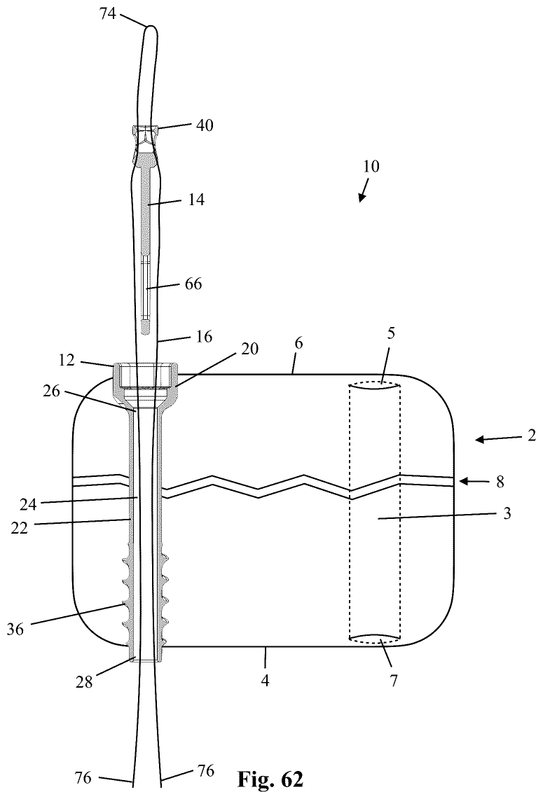

FIGS. 57-58 illustrate an example of a tension member 16 according to some embodiments of the present disclosure. As described herein throughout, the tension member 16 is an elongated flexible member having first and second ends 76, which are referred to herein as “free ends” 76, as illustrated by way of example in FIG. 57. In certain orientations of the tension member 16, a “looped end” 74 is created and differentiated from the “free ends” 76. As used herein throughout, the “looped end” 74 is not an actual end of the tension member 16, as these actual ends are the “free ends” 76, but instead the “looped end” 74 is created during assembly of the bone fracture fixation system 10 (and/or bone fracture fixation system 210 and/or bone fracture fixation system 410) when the tension member 16 is looped around an object or component, and/or makes a U-turn around an object or component, and/or is doubled over and/or folded over itself into an orientation in which both “free ends” 76 are on one end of the orientation, and the U-turned portion or “looped end” 74 is at the other end of the orientation, as illustrated by way of example in FIG. 58. By way of example, this orientation may be created when the tension member 16 is assembled with a locking element 14 (and/or locking element 214 and/or locking element 414) in situ by the surgeon or operating room staff during the fracture repair procedure or pre-assembled sometime prior to the surgical procedure. In some embodiments, the tension member 16 may be made of polyester, polyethylene, nitinol, or any suitable material. In some embodiments, the tension member 16 may be a surgical suture, tape, band, or any suitable tensionable component. In some embodiments, the tension member 16 may be round, flat, or a combination of round and flat.

Optionally, in any embodiment, the bone anchors described herein (e.g., bone anchor 12, 12′, 212, 212′, 412, 412′) may be partially threaded, fully threaded, or fully nonthreaded. Optionally, in any embodiments, the bone anchors described herein may be headed or non-headed. Optionally, in any embodiment, the bone anchors described herein may be made from any suitable medical grade metal or polymer, including but not limited to (and by way of example only) titanium, stainless steel, cobalt chrome, poly-ether-ether-ketone (PEEK), poly-ether-ketone-ketone (PEKK), and the like).

Optionally, in any embodiment, the distal extension 42 may include one or more open docking slots and no closed docking slots. Optionally, in any embodiment, the distal extension 42 may include one or more closed docking slots and no open docking slots.

By way of example, this method of repairing a patella fracture with an anchor system and tensioning members can also be performed using a Finger trap design, metal, PEEK, or a similar device which could be a button which may act as a base member for sutures to be organized on the base member using a finger trap technology or a finger trap technique which may include sutures woven in a manner where they crisscross or have a jacket of fibers around or adjoining the tensioning sutures. By way of example, once the construct is tensioned by pulling on the tensioning members or the free suture strands, the loop can reduce in size removing all the slack in the construct and providing a tension band effect for fixation of the patella fracture. The looped end may be transported through the cannulation within the screw in a crisscross manner to finally end up being connected to a base member or a component of the base member or a component of the screw which can provide an anchoring point for the user to create some form of a closed circuit which can be tensioned or reduced inside to remove the slack out of the system then act as barrier to any distraction of the fracture. The base member can be positioned adjoining or along the body of the screw, it may or may not have a press fit design. The circuit may begin with the maximum length desirable in the loop, which can then be reduced by reducing the size of the loop which can be reduced by pulling on the tension members which can be achieved by pulling on the tension members of the finger trap construct.

As used herein the terms “proximal” and “distal” are defined by the positioning of the head end of the bone anchor relative to the opposite end of the bone anchor. For example, the “proximal end” is defined as the end of the bone anchor 12 with the anchor head 20, and the “distal end” is defined as the end of the bone anchor 12 that is opposite of the end with the anchor head 20, regardless of the orientation of the bone anchor 12 during use. The “proximal direction” may be defined as the direction along a longitudinal axis extending through the proximal and distal ends from the distal end to the proximal end (and beyond). Similarly, the “distal direction” may be defined as the direction along a longitudinal axis extending through the proximal and distal ends from the proximal end to the distal end (and beyond). The term “proximally” can be defined as “in the proximal direction.” The term “distally” can be defined as “in the distal direction.”

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way but may also be configured in ways that are not listed.

Any of the features or attributes of the above-described embodiments and variations can be used in combination with any of the other features and attributes of the above-described embodiments and variations as desired. From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present disclosure and its practical application to thereby enable one of ordinary skill in the art to utilize the method and apparatus described herein in various embodiments and with various modifications as are suited to the particular use contemplated. All such modifications and variations are within the scope of the present disclosure as determined by the appended claims when interpreted in accordance with the benefit to which they are fairly, legally, and equitably entitled.

What is claimed is:

1. A bone fracture repair system, comprising:
a flexible tension member having first and second free ends and a flexible body extending between the first and second free ends, the flexible tension member oriented in an assembly configuration having a looped end comprising a U-shaped portion of the flexible body and an open end comprising the first and second free ends of the flexible tension member;
first and second bone anchors configured for implantation into bone, each of the first and second bone anchors having an anchor head at a proximal end, an elongated shank extending between the anchor head and a distal end, and a cannulation extending longitudinally through the elongated shaft and having a proximal opening at the proximal end and a distal opening at the distal end, the anchor head comprising a proximal recess including a circumferential mating surface positioned within the proximal recess;
a locking element having a proximal head and an elongated extension extending distally from the proximal head, the proximal head including a proximal opening, a pair of transverse openings, and a distal engagement surface, the elongated extension including a docking slot formed therein and configured to capture the looped end of the flexible tension member;
a first assembly configuration in which the locking element is associated with the first bone anchor, the looped end of the flexible tension member is positioned proximal of the locking element, the open end of the flexible tension member is positioned distal of the distal end of the first bone anchor, and the flexible body extends through the proximal opening of the locking element, the transverse openings of the locking element, and the cannulation of the first bone anchor; and
a second assembly configuration in which the elongated extension of the locking element extends into the cannulation of the first bone anchor, the looped end of the flexible tension member is captured by the docking slot of the locking element, the open end of the flexible tension member is positioned distal of the distal end of the first bone anchor, and the flexible body extends through the cannulation of the first bone anchor, the cannulation of the second bone anchor, and the proximal and transverse openings of the locking element before passing between the circumferential mating surface of the first bone anchor and the distal engagement surface of the locking element;
wherein the open end of the flexible tension member is configured to be pulled taut in the distal direction, thereby creating tension in the flexible tension member which causes the locking element to translate distally relative to the first bone anchor and transition the bone fracture repair system from an unlocked state that permits movement of the flexible tension member to a locked state that prohibits movement of the flexible tension member, where the locked state is maintained via compression and friction applied to the flexible tension member between the mating surface of the first bone anchor and the engagement surface of the locking element.

2. The bone fracture repair system of claim 1, wherein the proximal head of the locking element is configured to nest within the anchor head.

3. The bone fracture repair system of claim 1, wherein the distal engagement surface of the locking element includes at least one concave recess.

4. The bone fracture repair system of claim 1, wherein the second assembly configuration further comprises the flexible body extending distally from the looped end through the cannulation of the first bone anchor, then diagonally to the proximal end of the second bone anchor, then distally through the cannulation of the second bone anchor, then diagonally to the proximal head of the locking element, and then distally through the proximal opening of the locking element, the transverse openings of the locking element, and the cannulation of the first bone anchor.

5. The bone fracture repair system of claim 1, wherein the shanks of the first and second bone anchors have outer surfaces comprising a threaded portion and a non-threaded portion.

6. The bone fracture repair system of claim 1, wherein the locking element further includes at least one smooth curved surface bordering the docking slot.

7. The bone fracture repair system of claim 1, wherein docking slot is an open docking slot.

8. The bone fracture repair system of claim 1, wherein the docking slot has a hook configuration.

9. The bone fracture repair system of claim 1, wherein the docking slot extends to the distal end of the elongated extension.

10. The bone fracture repair system of claim 1, wherein the elongated extension is sized and configured to snugly nest within the cannulation of the first bone anchor to prevent pivoting movement of the locking element when the elongated extension is positioned within the cannulation of the first bone anchor.

11. A bone fracture repair system, comprising:
a flexible tension member having first and second free ends and a flexible body extending between the first and second free ends, the flexible tension member oriented in an assembly configuration having a looped end comprising a U-shaped portion of the flexible body and an open end comprising the first and second free ends of the flexible tension member;
a bone anchor configured for implantation into bone, the bone anchor having an anchor head at a proximal end, an elongated shank extending between the anchor head and a distal end, and a cannulation extending longitudinally through the elongated shaft and having a proximal opening at the proximal end and a distal opening at the distal end, the anchor head comprising a proximal recess including a circumferential mating surface positioned within the proximal recess;
a locking element having a proximal head and an elongated extension extending distally from the proximal head, the proximal head including a proximal opening, a pair of transverse openings, and a distal engagement surface, the elongated extension including a docking slot formed therein and configured to capture the looped end of the flexible tension member;
a first assembly configuration in which the locking element is associated with the bone anchor, the looped end of the flexible tension member is positioned proximal of the locking element, the open end of the flexible tension member is positioned distal of the distal end of the bone anchor, and the flexible body extends through the proximal opening of the locking element, the transverse openings of the locking element, and the cannulation of the bone anchor; and
a second assembly configuration in which the elongated extension of the locking element extends into the cannulation of the bone anchor, the looped end of the flexible tension member is captured by the docking slot of the locking element, the open end of the flexible tension member is positioned distal of the distal end of the bone anchor, and the flexible body extends through the cannulation of the bone anchor, a bone tunnel formed in the fractured bone under repair, and the proximal and transverse openings of the locking element before passing between the circumferential mating surface of the bone anchor and the distal engagement surface of the locking element;
wherein the open end of the flexible tension member is configured to be pulled taut in the distal direction, thereby creating tension in the flexible tension member which causes the locking element to translate distally relative to the bone anchor and transition the bone fracture repair system from an unlocked state that permits movement of the flexible tension member to a locked state that prohibits movement of the flexible tension member, where the locked state is maintained via compression and friction applied to the flexible tension member between the mating surface of the bone anchor and the engagement surface of the locking element.

12. The bone fracture repair system of claim 11, wherein the proximal head of the locking element is configured to nest within the anchor head.

13. The bone fracture repair system of claim 11, wherein the distal engagement surface of the locking element includes at least one concave recess.

14. The bone fracture repair system of claim 11, wherein the second assembly configuration further comprises the flexible body extending distally from the looped end through the cannulation of the first bone anchor, then diagonally to a proximal opening of the bone tunnel, then distally through the bone tunnel, then diagonally to the proximal head of the locking element, and then distally through the proximal opening of the locking element, the transverse openings of the locking element, and the cannulation of the bone anchor.

15. The bone fracture repair system of claim 11, wherein the shank of the bone anchor has an outer surface comprising a threaded portion and a non-threaded portion.

16. The bone fracture repair system of claim 11, wherein the locking element further includes at least one smooth curved surface bordering the docking slot.

17. The bone fracture repair system of claim 11, wherein docking slot is an open docking slot.

18. The bone fracture repair system of claim 11, wherein the docking slot has a hook configuration.

19. The bone fracture repair system of claim 11, wherein the docking slot extends to the distal end of the elongated extension.

20. The bone fracture repair system of claim 11, wherein the elongated extension is sized and configured to snugly nest within the cannulation of the bone anchor to prevent pivoting movement of the locking element when the elongated extension is positioned within the cannulation of the bone anchor.
